(19) United States
(12) Patent Application Publication
(10) Pub. No.: US 2024/0009201 A1
(43) Pub. Date: Jan. 11, 2024

(54) CYP11B2 BETA HYDROXYLASE INHIBITORS FOR HYPERTENSION

(71) Applicant: MINERALYS THERAPEUTICS, INC., San Francisco, CA (US)

(72) Inventors: Brian Taylor Slingsby, San Francisco, CA (US); William Connolly Watt, Seattle, WA (US); David Rodman, San Francisco, CA (US)

(73) Assignee: Mineralys Therapeutics, Inc., San Francisco, CA (US)

(21) Appl. No.: 18/250,402

(22) PCT Filed: Oct. 25, 2021
(86) PCT No.: PCT/US2021/056499

§ 371 Date: Apr. 25, 2023

Related U.S. Application Data
(60) Provisional application No. 63/223,719, filed on Jul. 20, 2021.
(60) Provisional application No. 63/105,745, filed on Oct. 26, 2020.

(51) Int. Cl. A61K 31/53; A61P 9/12

(52) U.S. Cl. CPC A61K 31/53; A61P 9/12

(57) ABSTRACT

The disclosure provides, inter alia, methods to treat hypertension in patients using CYP 11β2 beta hydroxylase inhibitors.

### BACKGROUND

Hypertension has a variety of etiologies. Due at least in part to this, the success of a pharmacological agent in treating one form of hypertension does not necessarily indicate that that agent will be successful in treating another form of hypertension. One major contributor to hypertension is the “renin cascade,” which culminates in the production of the potent vasoconstrictor angiotensin II. Renin is a protease which cleaves angiotensinogen to form angiotensin I, the latter which is then cleaved by a second enzyme (the angiotensin-converting enzyme or ACE) to form angiotensin II. Administration of a pharmacological agent which inhibits renin or ACE, or which antagonizes the angiotensin II end-product of the cascade, can lower blood pressure and provide a route for the treatment of this form of hypertension which affects a large portion of the hypertensive patient population.

Some individuals, however, have low levels of plasma-renin concentration or low plasma-renin activity, yet manifest hypertension. This form of hypertension is referred to as low renin hypertension. In these individuals, increased sodium intake is followed by an increase in blood pressure despite the fact that renin plasma concentrations are maintained or lowered. Agents active in treating essential hypertension, such as ACE inhibitors, are relatively ineffective in treating low renin hypertension. Thus, there is a need in the art for pharmacological agents that can effectively treat this form of hypertension. The disclosure is directed to this, as well as other, important ends.

### BRIEF SUMMARY

Provided herein are methods of treating hypertension in low renin hypertensive subjects in need thereof by administering to the low renin hypertensive subjects an effective amount of a CYP 11β2 beta hydroxylase inhibitor. In embodiments, the low renin hypertensive subject has primary aldosteronism. In embodiments, the low renin hypertensive subject has a plasma renin activity less than or equal to 0.6 units/milliliter/hour. In embodiments, the low renin hypertensive subject has a plasma aldosterone concentration of greater than or equal to 6 ng/dL as measured by an immunoassay such as ELISA or 1 ng/dL as measured by LC-MS. In embodiments, the low renin hypertensive subject is taking one or more hypertension medications (e.g., diuretics, ACE inhibitors, angiotensin receptor blockers, calcium channel blockers). In embodiments, the low renin hypertensive subject is not taking hypertension medications (e.g., diuretics, ACE inhibitors, angiotensin receptor blockers, calcium channel blockers). In embodiments, the CYP 11β2 beta hydroxylase inhibitor is a 1,2,4-triazine compound. In embodiments, the CYP 11β2 beta hydroxylase inhibitor is a compound of Formula (A) or a pharmaceutically acceptable salt thereof:

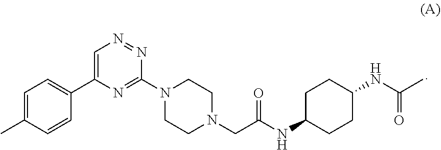 [SMILES: CC(=O)N[C@H]1CC[C@H](NC(=O)CN2CCN(c3nncc(-c4ccc(C)cc4)n3)CC2)CC1]

In embodiments, the CYP 11β2 beta hydroxylase inhibitor is a compound of Formula (A) in the form of a free base. In embodiments, the CYP 11β2 beta hydroxylase inhibitor is a pharmaceutically acceptable salt of the compound of Formula (A). In embodiments, the CYP 11β2 beta hydroxylase inhibitor is a monohydrobromide salt of the compound of Formula (A).

Provided herein are methods of identifying subjects for hypertension treatment with a CYP 11β2 beta hydroxylase inhibitor by: (i) measuring a systolic blood pressure of greater than 140 mmHg in the subject; (ii) measuring a diastolic blood pressure of greater than 90 mmHg in the subject; (iii) measuring a plasma renin activity less than or equal to 0.6 units/milliliter/hour in the subject; (iv) measuring a plasma aldosterone concentration of greater than or equal to 6 ng/dL in the subject or (v) a combination of two or more of the foregoing; thereby identifying the subject for hypertension treatment with a CYP 11β2 beta hydroxylase inhibitor. In embodiments, the subject has primary aldosteronism. In embodiments, the low renin hypertensive subject is taking one or more hypertension medications (e.g., diuretics, ACE inhibitors, angiotensin receptor blockers, calcium channel blockers). In embodiments, the CYP 11β2 beta hydroxylase inhibitor is a 1,2,4-triazine compound. In embodiments, the CYP 11β2 beta hydroxylase inhibitor is a compound of Formula (A) or a pharmaceutically acceptable salt thereof:

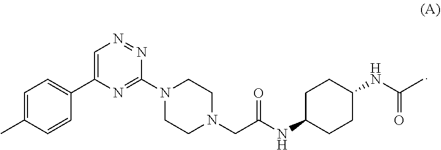 [SMILES: CC(=O)N[C@H]1CC[C@H](NC(=O)CN2CCN(c3nncc(-c4ccc(C)cc4)n3)CC2)CC1]

In embodiments, the CYP 11β2 beta hydroxylase inhibitor is a compound of Formula (A) in the form of a free base. In embodiments, the CYP 11β2 beta hydroxylase inhibitor is a pharmaceutically acceptable salt of the compound of Formula (A). In embodiments, the CYP 11β2 beta hydroxylase inhibitor is a monohydrobromide salt of the compound of Formula (A).

Provided here are methods of treating hypertension in a subject in need thereof by: (a) measuring: (i) a systolic blood pressure of greater than 140 mmHg or greater than 130 mmHg in the subject; (ii) a diastolic blood pressure of greater than 90 mmHg in the subject; (iii) a plasma renin activity less than or equal to 0.6 units/milliliter/hour in the subject; (iv) a plasma aldosterone concentration of greater than or equal to 6 ng/dL in the subject; or (v) a combination of two or more of the foregoing; and (b) administering to the subject an effective amount of a CYP 11β2 beta hydroxylase inhibitor. In embodiments, the low renin hypertensive subject has primary aldosteronism. In embodiments, the low renin hypertensive subject is taking one or more hypertension medications (e.g., diuretics, ACE inhibitors, angiotensin receptor blockers, calcium channel blockers). In embodiments, the CYP 11β2 beta hydroxylase inhibitor is a 1,2,4-triazine compound. In embodiments, the CYP 11β2 beta hydroxylase inhibitor is a compound of Formula (A) or a pharmaceutically acceptable salt thereof:

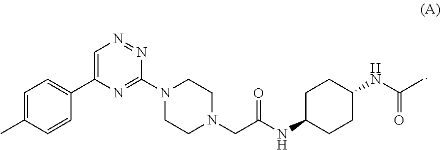 [SMILES: CC(=O)N[C@H]1CC[C@H](NC(=O)CN2CCN(c3nncc(-c4ccc(C)cc4)n3)CC2)CC1]

In embodiments, the CYP 11β2 beta hydroxylase inhibitor is a compound of Formula (A) in the form of a free base. In embodiments, the CYP 11β2 beta hydroxylase inhibitor is a pharmaceutically acceptable salt of the compound of Formula (A). In embodiments, the CYP 11β2 beta hydroxylase inhibitor is a monohydrobromide salt of the compound of Formula (A).

These and other embodiments and aspects of the disclosure are provided in detail herein.

### BRIEF DESCRIPTION OF THE DRAWINGS

FIG. 1: Measurement of serum aldosterone AUC-24 (h*pg/ml) in single ascending dose (SAD) study of the compound of Formula A in the form of an HBr salt.

FIG. 2: Measurement of serum cortisol AUC-24 (h*pg/ml) in single ascending dose (SAD) study of the compound of Formula A in the form of an HBr salt.

FIG. 3: Measurement of urine Na⁺ and Na⁺/K⁺ ratio single ascending dose (SAD) study of the compound of Formula A in the form of an HBr salt.

FIG. 4: Comparison of aldosterone levels (top panel) and compound of Formula A exposure (Cpu/Ki) (bottom panel).

FIG. 5: Dose effect of the compound of Formula A on duration of aldosterone suppression.

FIG. 6: Effect of the compound of Formula A exposure (bottom panel) on aldosterone levels (top panel) in multiple ascending dose (MAD) study.

FIG. 7: Effect on accumulation of 11-DOC at various dosage levels in multiple ascending dose (MAD) study.

FIG. 8: Dose effect of the compound of Formula A on duration of maximum aldosterone suppression.

FIG. 9: Dose effect of the compound of Formula A on Plasma Renin Activity (PRA).

FIG. 10: Physiological effect of the compound of Formula A on serum K⁺.

FIG. 11: Effect of various doses of the compound of Formula A on aldosterone secretion after adrenocorticotropic hormone (ACTH) stimulation.

FIG. 12: Effect of various doses of the compound of Formula A on various biomarkers.

FIG. 13: Telemetry protocol for low renin animal model experiment.

FIG. 14: Mean arterial blood pressure (MAP) in agouti yellow obese hyperleptinemic mice (Ay). Ay mice have approximately 5-8 mmHg elevation in MAP when compared to wild-type mice, due to direct leptin-mediated elevation in Cyp11β2 activity and aldosterone overproduction independent of RAS pathway activation (Hypertension, 2016 May; 67(5): 1020-1028; Circulation 2015 December 132(22); 2134-2145). Mice were treated with one of three Cyp11β2 inhibitors: the compound of formula (A) HBr as described herein; LCI699 or CIN-107. All three inhibitors reduced MAP to a similar degree and to a value comparable to that seen in control mice. The pooled data, comparing baseline to treated MAP in inhibitor-treated mice demonstrated statistically significant reduction (p=0.0022) verifying the ability of Cyp11β2 inhibitors to reduce blood pressure in the setting of aldosterone-mediated, Renin-angiotension-independent hypertension. There were no statistically significant differences in the treatment effect between the three inhibitors.

### DETAILED DESCRIPTION

### Definitions

The terms used throughout this specification are used in accordance with their plain and ordinary meaning within chemistry and biology. Unless defined otherwise, technical and scientific terms used herein have the same meaning as commonly understood by a person of ordinary skill in the art. See, e.g., Singleton et al., Dictionary of Microbiology and Molecular Biology, 2nd ed., J. Wiley & 20 Sons (New York, N.Y. 1994); Sambrook et al., Molecular Cloning, A Laboratory Manual, Cold Springs Harbor Press (Cold Springs Harbor, N.Y. 1989). Any methods, devices and materials similar or equivalent to those described herein can be used in the practice of this disclosure. The following definitions are provided to facilitate understanding of certain terms used frequently herein and are not meant to limit the scope of the claims or disclosure.

A “low renin hypertensive subject” or a “low renin hypertensive patient” refers to a subject who meets the following criteria: (i) has a plasma renin activity less than or equal to 0.6 units/milliliter/hour; (ii) has a plasma aldosterone concentration of greater than or equal to 6 ng/dL; (iii) has a systolic blood pressure of greater than 140 mmHg; (iv) has a diastolic blood pressure of greater than 90 mmHg; or (v) a combination of two or more of the foregoing. In embodiments, a low renin hypertensive subject meets all four criteria. In embodiments, the subject having low renin hypertensive subject has one or more of the above criteria. In embodiments, the subject having low renin hypertensive subject has a plasma renin activity less than or equal to 0.6 units/milliliter/hour. In embodiments, the subject is a low renin hypertensive subject. In embodiments, the low renin hypertensive subject has a plasma aldosterone concentration of greater than or equal to 6 ng/dL as measured by an immunoassay such as ELISA. In embodiments, the low renin hypertensive subject has a systolic blood pressure of greater than 140 mmHg. In embodiments, the low renin hypertensive subject has a systolic blood pressure of greater than 130 mmHg. In embodiments, the subject has elevated levels of aldosterone caused by autonomous aldosterone production. In embodiments, the subject does not have primary aldosteronism. In embodiments, the subject does not have clinically defined primary aldosteronism.

“CYP11β2” or “Cyp11B2” is a cytochrome P450 enzyme which catalyzes a series of reactions leading from 11-deoxycorticosterone (i.e., an aldosterone precursor) to aldosterone. Cyp11B2 is mainly expressed in an adrenal cortex spherical layer and a level of plasma aldosterone is regulated by enzymatic activity of Cyp11B2 present in the adrenal gland. Aldosterone is expressed in other tissues, such as cardiovascular, kidney, adipose, and brain.

An “inhibitor” refers to a compound (e.g. compounds described herein) that reduces activity when compared to a control, such as absence of the compound or a compound with known inactivity. An inhibitor can be a small molecule inhibitor, an antibody inhibitor, a protein inhibitor, a biomolecule inhibitor, a natural ligand, and the like. An “inhibitor” may be in the form of a pharmaceutically acceptable salt, e.g. of the compounds described herein.

A “protein inhibitor” refers to a compound that reduces protein activity when compared to a control, such as absence of the compound or a compound with known inactivity.

The term “inhibition,” “inhibit,” “inhibiting” and the like in reference to a protein-inhibitor interaction means negatively affecting (e.g. decreasing) the activity or function of the protein relative to the activity or function of the protein in the absence of the inhibitor. In embodiments, inhibition means negatively affecting (e.g. decreasing) the concentration or levels of the protein relative to the concentration or level of the protein in the absence of the inhibitor. In embodiments, inhibition refers to reduction of a disease or symptoms of disease. In embodiments, inhibition refers to a reduction in the activity of a particular protein target. Thus, inhibition includes, at least in part, partially or totally blocking stimulation, decreasing, preventing, or delaying activation, or inactivating, desensitizing, or down-regulating signal transduction or enzymatic activity or the amount of a protein. In embodiments, inhibition refers to a reduction of activity of a target protein resulting from a direct interaction (e.g. an inhibitor binds to the target protein). In embodiments, inhibition refers to a reduction of activity of a target protein from an indirect interaction (e.g. an inhibitor binds to a protein that activates the target protein, thereby preventing target protein activation).

The terms “inhibitor,” “repressor” or “antagonist” or “downregulator” interchangeably refer to a substance capable of detectably decreasing the expression or activity of a given gene or protein. The antagonist can decrease expression or activity 10%, 20%, 30%, 40%, 50%, 60%, 70%, 80%, 90% or more in comparison to a control in the absence of the antagonist. In embodiments, expression or activity is 2-fold, 5-fold, 10-fold or lower than the expression or activity in the absence of the antagonist.

The term “expression” includes any step involved in the production of the polypeptide including, but not limited to, transcription, post-transcriptional modification, translation, post-translational modification, and secretion. Expression can be detected using conventional techniques for detecting protein (e.g., ELISA, Western blotting, flow cytometry, immunofluorescence, immunohistochemistry, etc.).

A “CYP11β2 beta hydroxylase inhibitor” refers to a compound that reduces activity of CYP11β2 beta hydroxylase when compared to a control, such as absence of the compound or a compound with known inactivity. A CYP11β2 inhibitor reduces plasma aldosterone levels, urine aldosterone levels, and the like. The CYP11β2 beta hydroxylase inhibitors can be an antibody inhibitors, antisense nucleic acid inhibitors, aptamer inhibitors, small molecule inhibitors, natural ligands, protein inhibitors, biomolecule inhibitors, and the like. Exemplary CYP11β2 beta hydroxylase inhibitors are described in U.S. Pat. Nos. 10,029,993 and 10,329,263, the disclosures of which are incorporated by reference herein in their entirety. Other CYP11β2 beta hydroxylase inhibitors are described in U.S. Pat. No. 10,717,731 B2, the disclosures of which is also incorporated by reference herein in its entirety.

A compound of Formula (A) or a pharmaceutically acceptable salt thereof refers to N-[trans-4-(acetylamino)cyclohexyl]-2-{4-[5-(4-methylphenyl)-1,2,4-triazin-3-yl]piperazin-1-yl}acetamide or a pharmaceutically acceptable salt thereof, which is represented by Formula (A):

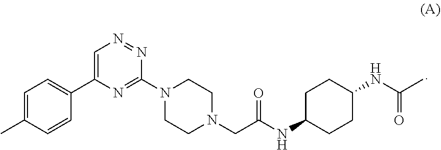 [SMILES: CC(=O)N[C@H]1CC[C@H](NC(=O)CN2CCN(c3nncc(-c4ccc(C)cc4)n3)CC2)CC1]

In embodiments, the compound of Formula (A) is in the form of a free base. In embodiments, the compound of Formula (A) is in the form of a pharmaceutically acceptable salt. In embodiments, the compound of Formula (A) is in the form of a monohydrobromide salt. In embodiments, the pharmaceutically acceptable salt of the compound of Formula (A) has the name N-[trans-4-(acetylamino)cyclohexyl]-2-{4-[5-(4-methylphenyl)-1,2,4-triazin-3-yl]piperazin-1-yl}acetamide monohydrobromide. The compound of Formula (A) and pharmaceutically salts thereof can be made by processes described, for example, in U.S. Pat. No. 10,029,993 and European Publication No. 3549935, the disclosures of which are incorporated by reference herein in their entirety.

As used herein, “Compound A HBr” refers to the hydrobromide salt of the compound of Formula (A).

The term “antibody” refers to a polypeptide encoded by an immunoglobulin gene or functional fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

The phrase “specifically (or selectively) binds” to an antibody or “specifically (or selectively) immunoreactive with,” when referring to a protein or peptide, refers to a binding reaction that is determinative of the presence of the protein, often in a heterogeneous population of proteins and other biologics. Thus, under designated immunoassay conditions, the specified antibodies bind to a particular protein at least two times the background and more typically more than 10 to 100 times background. Specific binding to an antibody under such conditions requires an antibody that is selected for its specificity for a particular protein. For example, polyclonal antibodies can be selected to obtain only a subset of antibodies that are specifically immunoreactive with the selected antigen and not with other proteins. This selection may be achieved by subtracting out antibodies that cross-react with other molecules. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Using Antibodies, A Laboratory Manual (1998) for a description of immunoassay formats and conditions that can be used to determine specific immunoreactivity).

An exemplary immunoglobulin (antibody) structural unit comprises a tetramer. Each tetramer is composed of two identical pairs of polypeptide chains, each pair having one “light” (about 25 kDa) and one “heavy” chain (about 50-70 kDa). The N-terminus of each chain defines a variable region of about 100 to 110 or more amino acids primarily responsible for antigen recognition. The terms “variable heavy chain,” “V_{H},” or “VH” refer to the variable region of an immunoglobulin heavy chain, including an Fv, scFv, dsFv or Fab; while the terms “variable light chain,” “V_{L}” or “VL” refer to the variable region of an immunoglobulin light chain, including of an Fv, scFv, dsFv or Fab.

Examples of antibody functional fragments include, but are not limited to, complete antibody molecules, antibody fragments, such as Fv, single chain Fv (scFv), complementarity determining regions (CDRs), VL (light chain variable region), VH (heavy chain variable region), Fab, F(ab)2′ and any combination of those or any other functional portion of an immunoglobulin peptide capable of binding to target antigen (see, e.g., Fundamental Immunology (Paul ed., 4th ed. 2001). As appreciated by one of skill in the art, various antibody fragments can be obtained by a variety of methods, for example, digestion of an intact antibody with an enzyme, such as pepsin; or de novo synthesis. Antibody fragments are often synthesized de novo either chemically or by using recombinant DNA methodology. Thus, the term antibody, as used herein, includes antibody fragments either produced by the modification of whole antibodies, or those synthesized de novo using recombinant DNA methodologies (e.g., single chain Fv) or those identified using phage display libraries (see, e.g., McCafferty et al., (1990) Nature 348:552). The term “antibody” also includes bivalent or bispecific molecules, diabodies, triabodies, and tetrabodies. Bivalent and bispecific molecules are described in, e.g., Kostelny et al. (1992) J. Immunol. 148:1547, Pack and Pluckthun (1992) Biochemistry 31:1579, Hollinger et al. (1993), PNAS. USA 90:6444, Gruber et al. (1994) J Immunol. 152:5368, Zhu et al. (1997) Protein Sci. 6:781, Hu et al. (1996) Cancer Res. 56:3055, and Adams et al. (1993) Cancer Res. 53:4026.

An “antisense nucleic acid” as referred to herein is a nucleic acid (e.g., DNA or RNA molecule) that is complementary to at least a portion of a specific target nucleic acid and is capable of reducing transcription of the target nucleic acid (e.g. mRNA from DNA), reducing the translation of the target nucleic acid (e.g. mRNA), altering transcript splicing (e.g. single stranded morpholino oligo), or interfering with the endogenous activity of the target nucleic acid. See, e.g., Weintraub, Scientific American, 262:40 (1990). Typically, synthetic antisense nucleic acids (e.g. oligonucleotides) are generally between 15 and 25 bases in length. Thus, antisense nucleic acids are capable of hybridizing to (e.g. selectively hybridizing to) a target nucleic acid. In embodiments, the antisense nucleic acid hybridizes to the target nucleic acid in vitro. In embodiments, the antisense nucleic acid hybridizes to the target nucleic acid in a cell. In embodiments, the antisense nucleic acid hybridizes to the target nucleic acid in an organism. In embodiments, the antisense nucleic acid hybridizes to the target nucleic acid under physiological conditions. Antisense nucleic acids may comprise naturally occurring nucleotides or modified nucleotides.

In the cell, the antisense nucleic acids hybridize to the corresponding RNA forming a double-stranded molecule. The antisense nucleic acids interfere with the endogenous behavior of the RNA and inhibit its function relative to the absence of the antisense nucleic acid. Furthermore, the double-stranded molecule may be degraded via the RNAi pathway. The use of antisense methods to inhibit the in vitro translation of genes is well known in the art (Marcus-Sakura, Anal. Biochem., 172:289, (1988)). Further, antisense molecules which bind directly to the DNA may be used. Antisense nucleic acids may be single or double stranded nucleic acids. Non-limiting examples of antisense nucleic acids include siRNAs (including their derivatives or pre-cursors, such as nucleotide analogs), short hairpin RNAs (shRNA), micro RNAs (miRNA), saRNAs (small activating RNAs) and small nucleolar RNAs (snoRNA) or certain of their derivatives or pre-cursors.

The term “aptamer” as provided herein refers to oligonucleotides (e.g. short oligonucleotides or deoxyribonucleotides), that bind (e.g. with high affinity and specificity) to proteins, peptides, and small molecules. Aptamers typically have defined secondary or tertiary structure owing to their propensity to form complementary base pairs and, thus, are often able to fold into diverse and intricate molecular structures. The three-dimensional structures are essential for aptamer binding affinity and specificity, and specific three-dimensional interactions drives the formation of aptamer-target complexes. Aptamers can be selected in vitro from very large libraries of randomized sequences by the process of systemic evolution of ligands by exponential enrichment (SELEX as described in Ellington A D, Szostak J W (1990) In vitro selection of RNA molecules that bind specific ligands. Nature 346:818-822; Tuerk C, Gold L (1990) Systematic evolution of ligands by exponential enrichment: RNA ligands to bacteriophage T4 DNA polymerase. Science 249:505-510) or by developing SOMAmers (slow off-rate modified aptamers) (Gold L et al. (2010) Aptamer-based multiplexed proteomic technology for biomarker discovery. PLoS ONE 5(12):e15004). Applying the SELEX and the SOMAmer technology includes for instance adding functional groups that mimic amino acid side chains to expand the aptamer's chemical diversity. As a result high affinity aptamers for almost any protein target are enriched and identified. Aptamers exhibit many desirable properties for targeted drug delivery, such as ease of selection and synthesis, high binding affinity and specificity, flexible structure, low immunogenicity, and versatile synthetic accessibility.

The term “oligonucleotide,” “nucleic acid,” “nucleic acid molecule,” “nucleic acid oligomer,” “nucleic acid sequence,” “nucleic acid fragment” and “polynucleotide” are used interchangeably and are intended to include, but are not limited to, a polymeric form of nucleotides covalently linked together that may have various lengths, either deoxyribonucleotides or ribonucleotides, or analogs, derivatives or modifications thereof. Different polynucleotides may have different three-dimensional structures, and may perform various functions, known or unknown. Non-limiting examples of polynucleotides include a gene, a gene fragment, an exon, an intron, intergenic DNA (including, without limitation, heterochromatic DNA), messenger RNA (mRNA), transfer RNA, ribosomal RNA, a ribozyme, cDNA, a recombinant polynucleotide, a branched polynucleotide, a plasmid, a vector, isolated DNA of a sequence, isolated RNA of a sequence, a nucleic acid probe, and a primer. Oligonucleotides useful in the methods of the disclosure may comprise natural nucleic acid sequences and variants thereof, artificial nucleic acid sequences, or a combination of such sequences.

The term “natural ligand” is a molecule which displays specific reactivity for (i.e., specifically recognizes and binds to) a target molecule (e.g., a protein). A ligand for a receptor means any compound that binds to the receptor and thereby modulates the natural function of the receptor. The term includes peptide, modified peptide, polypeptide, protein and small molecule ligands, such as synthetic chemical compounds, naturally occurring compounds or small organic molecules. Alternatively, the ligand may be an antibody or antibody fragment, or a nucleic acid or nucleic acid derived material.

The term “small molecule” refers to a compound having molecular mass of less than 3000 Daltons. A “small organic molecule” is a small molecule that comprises carbon.

“Contacting” is used in accordance with its plain ordinary meaning and refers to the process of allowing at least two distinct species (e.g. chemical compounds including biomolecules or cells) to become sufficiently proximal to react, interact or physically touch. It should be appreciated; however, the resulting reaction product can be produced directly from a reaction between the added reagents or from an intermediate from one or more of the added reagents that can be produced in the reaction mixture. The term “contacting” may include allowing two species to react, interact, or physically touch, wherein the two species may be a compound as described herein and a protein or enzyme. In embodiments contacting includes allowing a compound described herein to interact with a protein or enzyme that is involved in a signaling pathway.

The term “aberrant” as used herein refers to different from normal. When used to describe enzymatic activity or protein function, aberrant refers to activity or function that is greater or less than a normal control or the average of normal non-diseased control samples. Aberrant activity may refer to an amount of activity that results in a disease, wherein returning the aberrant activity to a normal or non-disease-associated amount (e.g. by administering a compound or using a method as described herein), results in reduction of the disease or one or more disease symptoms.

The term “pharmaceutically acceptable salts” is meant to include salts of the active compounds that are prepared with relatively nontoxic acids or bases, depending on the particular substituents found on the compounds described herein. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, monohydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, oxalic, methanesulfonic, and the like. Also included are salts of amino acids such as arginate and the like, and salts of organic acids like glucuronic or galactunoric acids and the like (see, for example, Berge et al., Journal of Pharmaceutical Science, 66:1-19 (1977)). Certain compounds of the present disclosure contain both basic and acidic functionalities that allow the compounds to be converted into either base or acid addition salts.

Thus, the compounds of the present disclosure may exist as salts, such as with pharmaceutically acceptable acids. The present disclosure includes such salts. Non-limiting examples of such salts include hydrochlorides, hydrobromides, phosphates, sulfates, methanesulfonates, nitrates, maleates, acetates, citrates, fumarates, propionates, tartrates (e.g., (+)-tartrates, (−)-tartrates, or mixtures thereof including racemic mixtures), succinates, benzoates, and salts with amino acids such as glutamic acid, and quaternary ammonium salts (e.g. methyl iodide, ethyl iodide, and the like). These salts may be prepared by methods known in the art.

The neutral forms of the compounds can be regenerated by contacting the salt with a base or acid and isolating the parent compound in the conventional manner. The parent form of the compound may differ from the various salt forms in certain physical properties, such as solubility in polar solvents.

In addition to salt forms, the present disclosure provides compounds, which are in a prodrug form. Prodrugs of the compounds described herein are those compounds that readily undergo chemical changes under physiological conditions to provide the compounds of the present disclosure. Prodrugs of the compounds described herein may be converted in vivo after administration. Additionally, prodrugs can be converted to the compounds of the present disclosure by chemical or biochemical methods in an ex vivo environment, such as, for example, when contacted with a suitable enzyme or chemical reagent.

Certain compounds of the disclosure can exist in unsolvated forms as well as solvated forms, including hydrated forms. In general, the solvated forms are equivalent to unsolvated forms and are encompassed within the scope of the present disclosure. Certain compounds may exist in multiple crystalline or amorphous forms. In general, all physical forms are equivalent for the uses contemplated herein and are intended to be within the scope of the present disclosure.

“Pharmaceutically acceptable excipient” and “pharmaceutically acceptable carrier” refer to a substance that aids the administration of an active agent to and absorption by a subject and can be included in the compositions of the present disclosure without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the disclosure. One of skill in the art will recognize that other pharmaceutical excipients are useful in the present disclosure.

The term “about” means a range of values including the specified value, which a person of ordinary skill in the art would consider reasonably similar to the specified value. In embodiments, about means within a standard deviation using measurements generally acceptable in the art. In embodiments, about means a range extending to +/−10% of the specified value. In embodiments, about includes the specified value.

“Patient” or “subject in need thereof” refers to a living organism suffering from or prone to a disease or condition that can be treated by administration of a compound or pharmaceutical composition as provided herein. Non-limiting examples include humans, other mammals, bovines, rats, mice, dogs, monkeys, goat, sheep, cows, deer, and other non-mammalian animals. In embodiments, a patient is human.

The terms “treating”, or “treatment” refers to any indicia of success in the therapy or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. The term “treating” and conjugations thereof, may include prevention of an injury, pathology, condition, or disease. In embodiments, treating is preventing. In embodiments, treating does not include preventing.

“Treating” or “treatment” as used herein (and as well-understood in the art) also broadly includes any approach for obtaining beneficial or desired results in a subject's condition, including clinical results. Beneficial or desired clinical results can include, but are not limited to, alleviation or amelioration of one or more symptoms or conditions, diminishment of the extent of a disease, stabilizing (i.e., not worsening) the state of disease, prevention of a disease's transmission or spread, delay or slowing of disease progression, amelioration or palliation of the disease state, diminishment of the reoccurrence of disease, and remission, whether partial or total and whether detectable or undetectable. In other words, “treatment” as used herein includes any cure, amelioration, or prevention of a disease. Treatment may prevent the disease from occurring; inhibit the disease's spread; relieve the disease's symptoms, fully or partially remove the disease's underlying cause, shorten a disease's duration, or do a combination of these things.

“Treating” and “treatment” as used herein include prophylactic treatment. Treatment methods include administering to a subject a therapeutically effective amount of an active agent. The administering step may be a single administration or may include a series of administrations. The length of the treatment period depends on a variety of factors, such as the severity of the condition, the age of the patient, the concentration of active agent, the activity of the compositions used in the treatment, or a combination thereof. It will also be appreciated that the effective dosage of an agent used for the treatment or prophylaxis may increase or decrease over the course of a particular treatment or prophylaxis regime. Changes in dosage may result and become apparent by standard diagnostic assays known in the art. In embodiments, chronic administration may be required. For example, the compositions are administered to the subject in an amount and for a duration sufficient to treat the patient. In embodiments, the treating or treatment is not prophylactic treatment.

The term “prevent” refers to a decrease in the occurrence of disease symptoms in a patient. As indicated above, the prevention may be complete (no detectable symptoms) or partial, such that fewer symptoms are observed than would likely occur absent treatment.

A “effective amount” is an amount sufficient for a compound to accomplish a stated purpose relative to the absence of the compound (e.g. achieve the effect for which it is administered, treat a disease, reduce enzyme activity, increase enzyme activity, reduce a signaling pathway, or reduce one or more symptoms of a disease or condition). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. An “activity decreasing amount,” as used herein, refers to an amount of antagonist required to decrease the activity of an enzyme relative to the absence of the antagonist. A “function disrupting amount,” as used herein, refers to the amount of antagonist required to disrupt the function of an enzyme or protein relative to the absence of the antagonist. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

For any compound described herein, the therapeutically effective amount can be initially determined from cell culture assays. Target concentrations will be those concentrations of active compound(s) that are capable of achieving the methods described herein, as measured using the methods described herein or known in the art.

As is well known in the art, therapeutically effective amounts for use in humans can also be determined from animal models. For example, a dose for humans can be formulated to achieve a concentration that has been found to be effective in animals. The dosage in humans can be adjusted by monitoring compounds effectiveness and adjusting the dosage upwards or downwards, as described above. Adjusting the dose to achieve maximal efficacy in humans based on the methods described above and other methods is well within the capabilities of the ordinarily skilled artisan.

The term “therapeutically effective amount,” as used herein, refers to that amount of the therapeutic agent sufficient to ameliorate the disorder, as described above. For example, for the given parameter, a therapeutically effective amount will show an increase or decrease of at least 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a control.

Dosages may be varied depending upon the requirements of the patient and the compound being employed. The dose administered to a patient, in the context of the present disclosure, should be sufficient to effect a beneficial therapeutic response in the patient over time. The size of the dose also will be determined by the existence, nature, and extent of any adverse side-effects. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. Dosage amounts and intervals can be adjusted individually to provide levels of the administered compound effective for the particular clinical indication being treated. This will provide a therapeutic regimen that is commensurate with the severity of the individual's disease state.

As used herein, the term “administering” means oral administration, administration as a suppository, topical contact, intravenous, parenteral, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of administration include, but are not limited to, the use of liposomal formulations, intravenous infusion, transdermal patches, etc. In embodiments, administering does not include administration of any active agent other than the recited active agent. “Co-administer” it is meant that a composition described herein is administered at the same time, just prior to, or just after the administration of one or more additional therapies. The compounds provided herein can be administered alone or can be coadministered to the patient. Coadministration is meant to include simultaneous or sequential administration of the compounds individually or in combination (more than one compound). Thus, the compositions can also be combined, when desired, with other active substances (e.g. to reduce metabolic degradation).

“Control” or “control experiment” is used in accordance with its plain ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects. In some embodiments, a control is the measurement of the activity of a protein in the absence of a compound as described herein (including embodiments and examples).

A “hypertension medication” refers to any medication that can treat hypertension. Exemplary hypertension medications include diuretics, ACE inhibitors, angiotensin receptor blockers, calcium channel blockers, and the like.

A “diuretic” refers to a hypertension medication that increases the production of urine, thereby increasing the amount of water and salt eliminated from the body. The diuretic can be a carbonic anhydrase inhibitor, a loop diuretic, a potassium-sparing diuretic, a thiazide diuretic, or any other diuretic known in the art. Exemplary carbonic anhydrase inhibitors include acetazolamide, brinzolamide, dorzolamide, dichlorphenamide, ethoxaolamide, zoniamide, indisulam, and methazolamide. Exemplary loop diuretics include bumatenide, ethacrynic acid, torsemide, and furosemide. Exemplary potassium-sparing diuretics include epelerenone, triamterene, spironolactone, and amiloride. Exemplary thiazide diuretics include indapamide, hydrochlorothiazide, chlorthalidone, metolazone, methyclothiazide, chlorothiazide, methylclothiazide, metolazone, bendroflumethiazide, polythiazide, and hydroflumethiazide. Other diuretics include pamabrom and mannitol.

An “angiotensin-converting enzyme inhibitor” or “ACE inhibitor” refers to a hypertension medication that block angiotensin I from being converted to angiotensin II, thereby dilating blood vessels and lowering blood pressure. Exemplary ACE inhibitors include benazepril, zofenopril, perindopril, trandolapril, captopril, enalapril, lisinopril, and ramipril.

An “angiotensin receptor blocker” or “angiotensin II inhibitor” refers to a hypertension medication that blocks the receptor binding of angiotensin II, thereby dilating blood vessels and lowering blood pressure. Exemplary angiotensin receptor blockers include eprosartan, olmesartan, valsartan, candesartan, losartan, telmisartan, irbesartan, valsartan, and azilsartan medoxomil.

A “calcium channel blocker” refers to hypertension medication that can block calcium from entering the cells of the heart and arteries via a calcium channel, thereby lowering blood pressure. A calcium channel blocker can be a dihydropyridine calcium channel blocker, a phenylalkylamine calcium channel blocker, a benzothiazepine calcium channel blocker, a nonselective calcium channel blocker, or any other calcium channel blocker known in the art. Dihydropyridine calcium channel blockers include amoldipine, aranidipine, azelnidipine, barnidipine, benidipine, cilnidine, clevidipine, efonidipine, felodipine, isradipine, lacidipine, lercanidipine, manidipine, nicardipine, nifedipine, nilvadipine, nimodipine, nisoldipine, nitrendipine, and pranidipine. Phenylalkylamine calcium channel blockers include fendiline, gallipamil, and verapamil. Benzothiazepine calcium channel blockers include diltiazem. Nonselective calcium channel blockers include mibefradil, bepridil, flunarizine, fluspirilene, and fendiline. Other calcium channel blockers include gabapentin, pregabalin, and ziconotide.

Methods of Treatment

Provided herein are methods of treating hypertension in a low renin hypertensive subject in need thereof by administering to the subject an effective amount of a CYP11β2 beta hydroxylase inhibitor. In embodiments, the methods comprise treating hypertension in a low renin hypertensive subject. In embodiments, the CYP 11β2 beta hydroxylase inhibitor is selective for inhibition of CYP 11β2 beta hydroxylase activity relative to inhibition of CYP11β2 beta hydroxylase activity, preferably wherein the inhibition constant (Ki) for CYP11β2 beta hydroxylase divided by the Ki for CYP 11β2 beta hydroxylase is greater than 100. In embodiments, the CYP11β2 beta hydroxylase inhibitor is a compound described in U.S. Pat. No. 10,029,993, the disclosure of which is incorporated by reference herein. In embodiments, the CYP11β2 beta hydroxylase inhibitor is a compound described in U.S. Pat. No. 10,329,263, the disclosure of which is incorporated by reference herein. In embodiments, the CYP11B2 beta hydroxylase inhibitor is a 1,2,4-triazine compound or a pharmaceutically acceptable salt thereof. In embodiments, the CYP11β2 beta hydroxylase inhibitor is a compound of Formula (A) or a pharmaceutically acceptable salt thereof:

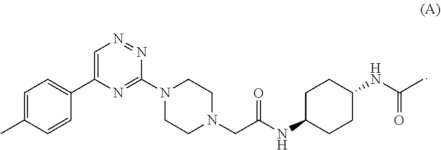 [SMILES: CC(=O)N[C@H]1CC[C@H](NC(=O)CN2CCN(c3nncc(-c4ccc(C)cc4)n3)CC2)CC1]

In embodiments, the CYP11β2 beta hydroxylase inhibitor is a pharmaceutically acceptable salt of the compound of Formula (A). In embodiments, the CYP11β2 beta hydroxylase inhibitor is a monohydrobromide salt of the compound of Formula (A). In embodiments, the CYP11β2 beta hydroxylase inhibitor is the free base form of the compound of Formula (A). In embodiments, the CYP11β2 beta hydroxylase inhibitor is an antibody (e.g., antibody inhibitor). In embodiments, the CYP11β2 beta hydroxylase inhibitor is an antisense nucleic acid (e.g., antisense nucleic acid inhibitor). In embodiments, the CYP11β2 beta hydroxylase inhibitor is an aptamer (e.g., aptamer inhibitor). In embodiments, the CYP11β2 beta hydroxylase inhibitor is a small molecule (e.g., small molecule inhibitor). In embodiments, the CYP11β2 beta hydroxylase inhibitor is a natural ligand, a protein inhibitor, a biomolecule inhibitor, or the like.

In embodiments of the methods described herein, the CYP 11β2 beta hydroxylase inhibitor is a compound of formula (I) or a pharmaceutically acceptable salt thereof:

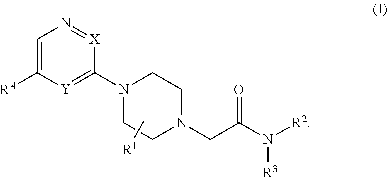

1) wherein X and Y represent any of the following (i) to (iii):
   (i) X is N, and Y is CH or C—RY,
   (ii) X is CH, and Y is N, or
   (iii) X is CH, and Y is CH;
2) R^{Y} represents an alkyl group;
3) R^{A} represents a cycloalkyl group which may be substituted, a cycloalkenyl group which may be substituted, an aryl group which may be substituted, or a 6- to 10-membered monocyclic or bicyclic heteroaryl group which may be partially hydrogenated and may be substituted;
4) R¹ represents a hydrogen atom, or an alkyl group;
5) R² represents an alkyl group which may be substituted, a cycloalkyl group which may be substituted, an aliphatic heterocyclic group which may be substituted, or a heteroaryl group which may be partially hydrogenated and may be substituted; and
6) R³ represents a hydrogen atom, or an alkyl group, or a pharmaceutically acceptable salt thereof.

In embodiments, the low renin hypertensive subject: (i) is taking or has taken a hypertension medication; (ii) has a plasma renin activity less than or equal to 0.6 units/milliliter/hour; (iii) has a plasma aldosterone concentration of greater than or equal to 6 ng/dL as measured by an immunoassay such as ELISA or 1 ng/dL as measured by LC-MS; or (iv) a combination of two or more thereof. In embodiments, the low renin hypertensive subject: (i) is taking or has taken a hypertension medication; (ii) has a plasma renin activity less than or equal to 0.6 units/milliliter/hour; and (iii) has a plasma aldosterone concentration of greater than or equal to 6 ng/dL as measured by an immunoassay such as ELISA or 1 ng/dL as measured by LC-MS. In embodiments, the low renin hypertensive subject: (i) is taking or has taken a hypertension medication; (ii) has a plasma renin activity less than or equal to 0.6 units/milliliter/hour when not taking a hypertension medication; and (iii) has a plasma aldosterone concentration of greater than or equal to 6 ng/dL as measured by an immunoassay such as ELISA or 1 ng/dL as measured by LC-MS. In embodiments, the low renin hypertensive subject: (i) is taking or has taken a hypertension medication; (ii) has a plasma renin activity less than or equal to 1 units/milliliter/hour; and (iii) has a plasma aldosterone concentration of greater than or equal to 6 ng/dL as measured by an immunoassay such as ELISA or 1 ng/dL as measured by LC-MS.

In embodiments, the low renin hypertensive subject: (i) is taking a hypertension medication; (ii) has a plasma renin activity less than or equal to 0.6 units/milliliter/hour; (iii) has a plasma aldosterone concentration of greater than or equal to 6 ng/dL as measured by an immunoassay such as ELISA or 1 ng/dL as measured by LC-MS; or (iv) a combination of two or more thereof. In embodiments, the low renin hypertensive subject: (i) is taking a hypertension medication; (ii) has a plasma renin activity less than or equal to 0.6 units/milliliter/hour; and (iii) has a plasma aldosterone concentration of greater than or equal to 6 ng/dL as measured by an immunoassay such as ELISA or 1 ng/dL as measured by LC-MS. In embodiments, the low renin hypertensive subject: (i) is taking a hypertension medication; (ii) has a plasma renin activity less than or equal to 0.6 units/milliliter/hour when not taking a hypertension medication; and (iii) has a plasma aldosterone concentration of greater than or equal to 6 ng/dL as measured by an immunoassay such as ELISA or 1 ng/dL as measured by LC-MS. In embodiments, the low renin hypertensive subject: (i) is taking a hypertension medication; (ii) has a plasma renin activity less than or equal to 1 units/milliliter/hour; and (iii) has a plasma aldosterone concentration of greater than or equal to 6 ng/dL as measured by an immunoassay such as ELISA or 1 ng/dL as measured by LC-MS.

In embodiments, the low renin hypertensive subject: (i) is taking or has taken two hypertension medications; (ii) has a plasma renin activity less than or equal to 0.6 units/milliliter/hour; (iii) has a plasma aldosterone concentration of greater than or equal to 6 ng/dL as measured by an immunoassay such as ELISA or 1 ng/dL as measured by LC-MS; or (iv) a combination of two or more thereof. In embodiments, the low renin hypertensive subject: (i) is taking two hypertension medications; (ii) has a plasma renin activity less than or equal to 0.6 units/milliliter/hour; and (iii) has a plasma aldosterone concentration of greater than or equal to 6 ng/dL as measured by an immunoassay such as ELISA or 1 ng/dL as measured by LC-MS. In embodiments, the low renin hypertensive subject: (i) is taking or has taken two hypertension medications; (ii) has a plasma renin activity less than or equal to 0.6 units/milliliter/hour when not taking a hypertension medication; and (iii) has a plasma aldosterone concentration of greater than or equal to 6 ng/dL as measured by an immunoassay such as ELISA or 1 ng/dL as measured by LC-MS. In embodiments, the low renin hypertensive subject: (i) is taking or has taken two hypertension medications; (ii) has a plasma renin activity less than or equal to 1 units/milliliter/hour; and (iii) has a plasma aldosterone concentration of greater than or equal to 6 ng/dL as measured by an immunoassay such as ELISA or 1 ng/dL as measured by LC-MS. In embodiments, the low renin hypertensive subject is taking or has taken three or four hypertension medications.

In embodiments, the low renin hypertensive subject: (i) is taking two hypertension medications; (ii) has a plasma renin activity less than or equal to 0.6 units/milliliter/hour; (iii) has a plasma aldosterone concentration of greater than or equal to 6 ng/dL as measured by an immunoassay such as ELISA or 1 ng/dL as measured by LC-MS; or (iv) a combination of two or more thereof. In embodiments, the low renin hypertensive subject: (i) is taking two hypertension medications; (ii) has a plasma renin activity less than or equal to 0.6 units/milliliter/hour; and (iii) has a plasma aldosterone concentration of greater than or equal to 6 ng/dL as measured by an immunoassay such as ELISA or 1 ng/dL as measured by LC-MS. In embodiments, the low renin hypertensive subject: (i) is taking two hypertension medications; (ii) has a plasma renin activity less than or equal to 0.6 units/milliliter/hour when not taking a hypertension medication; and (iii) has a plasma aldosterone concentration of greater than or equal to 6 ng/dL as measured by an immunoassay such as ELISA or 1 ng/dL as measured by LC-MS. In embodiments, the low renin hypertensive subject: (i) is taking two hypertension medications; (ii) has a plasma renin activity less than or equal to 1 units/milliliter/hour; and (iii) has a plasma aldosterone concentration of greater than or equal to 6 ng/dL as measured by an immunoassay such as ELISA or 1 ng/dL as measured by LC-MS. In embodiments, the low renin hypertensive subject or the low renin hypertensive subject is taking three or four hypertension medications.

In embodiments, the low renin hypertensive subject: (i) has a plasma renin activity less than or equal to 0.6 units/milliliter/hour; (ii) has a plasma aldosterone concentration of greater than or equal to 6 ng/dL as measured by an immunoassay such as ELISA or 1 ng/dL as measured by LC-MS; or (iii) a combination thereof. In embodiments, the low renin hypertensive subject: (i) has a plasma renin activity less than or equal to 1 units/milliliter/hour; (ii) has a plasma aldosterone concentration of greater than or equal to 6 ng/dL as measured by an immunoassay such as ELISA or 1 ng/dL as measured by LC-MS; or (iii) a combination thereof.

In embodiments, the low renin hypertensive subject: (i) has a plasma renin activity less than or equal to 0.6 units/milliliter/hour; (ii) has a plasma aldosterone concentration of greater than or equal to 6 ng/dL as measured by an immunoassay such as ELISA or 1 ng/dL as measured by LC-MS; (iii) has a systolic blood pressure of greater than 140 mmHg; (iv) has a diastolic blood pressure of greater than 90 mmHg; or (v) a combination of two or more of the foregoing. In embodiments, the low renin hypertensive subject: (i) has a plasma renin activity less than or equal to 0.6 units/milliliter/hour; (ii) has a plasma aldosterone concentration of greater than or equal to 6 ng/dL as measured by an immunoassay such as ELISA or 1 ng/dL as measured by LC-MS; (iii) has a systolic blood pressure of greater than 140 mmHg; and (iv) has a diastolic blood pressure of greater than 90 mmHg. In embodiments, the low renin hypertensive subject: (i) has a plasma renin activity less than or equal to 0.6 units/milliliter/hour when not taking a hypertension medication or less than or equal to 1 units/milliliter/hour when taking a hypertension medication; (ii) has a plasma aldosterone concentration of greater than or equal to 6 ng/dL as measured by an immunoassay such as ELISA or 1 ng/dL as measured by LC-MS; (iii) has a systolic blood pressure of greater than 130 mmHg; (iv) has a diastolic blood pressure of greater than 90 mmHg; or (v) a combination of two or more of the foregoing. In embodiments, the low renin hypertensive subject: (i) has a plasma renin activity less than or equal to 0.6 units/milliliter/hour when not taking a hypertension medication or less than or equal to 1 units/milliliter/hour when taking a hypertension medication; (ii) has a plasma aldosterone concentration of greater than or equal to 6 ng/dL as measured by an immunoassay such as ELISA or 1 ng/dL as measured by LC-MS; (iii) has a systolic blood pressure of greater than 130 mmHg; and (iv) has a diastolic blood pressure of greater than 90 mmHg.

In embodiments, the low renin hypertensive subject: (i) is taking or has taken a hypertension medications; (ii) has a plasma renin activity less than or equal to 0.6 units/milliliter/hour; (iii) has a plasma aldosterone concentration of greater than or equal to 6 ng/dL as measured by an immunoassay such as ELISA or 1 ng/dL as measured by LC-MS; (iv) has a systolic blood pressure of greater than 140 mmHg; (v) has a diastolic blood pressure of greater than 90 mmHg, or (vi) a combination of two or more thereof. In embodiments, the subject is taking a hypertension medication. In embodiments, the subject is taking two hypertension medications. In embodiments, the low renin hypertensive subject: (i) is taking or has taken a hypertension medication; (ii) has a plasma renin activity less than or equal to 0.6 units/milliliter/hour when not taking a hypertension medication or less than or equal to 1 units/milliliter/hour when taking a hypertension medication; (iii) has a plasma aldosterone concentration of greater than or equal to 6 ng/dL as measured by an immunoassay such as ELISA or 1 ng/dL as measured by LC-MS; (iv) has a systolic blood pressure of greater than 130 mmHg; (v) has a diastolic blood pressure of greater than 90 mmHg, or (vi) a combination of two or more thereof. In embodiments, the subject is taking a hypertension medication. In embodiments, the subject is taking two hypertension medications.

In embodiments of the methods described herein, the subject is taking or has taken a hypertension medication. In embodiments, the subject is taking a hypertension medication. In embodiments, the subject is taking or has taken two hypertension medications. In embodiments, the subject is taking two hypertension medications. The hypertension medication can be any known in the art. In embodiments, the hypertension medication is a diuretic, an ACE inhibitor, an angiotensin receptor blocker, a calcium channel blocker, or a combination of two or more thereof. In embodiments, the diuretic is a carbonic anhydrase inhibitor, a loop diuretic, a potassium-sparing diuretic, or a thiazide diuretic. In embodiments, the diuretic is acetazolamide, brinzolamide, dorzolamide, dichlorphenamide, ethoxaolamide, zoniamide, indisulam, methazolamide, bumatenide, ethacrynic acid, torsemide, furosemide, epelerenone, triamterene, spironolactone, amiloride, indapamide, hydrochlorothiazide, chlorthalidone, metolazone, methyclothiazide, chlorothiazide, methylclothiazide, metolazone, bendroflumethiazide, polythiazide, hydroflumethiazide, or a combination of two or more thereof. In embodiments, the ACE inhibitor is benazepril, zofenopril, perindopril, trandolapril, captopril, enalapril, lisinopril, ramipril, or a combination of two or more thereof. In embodiments, the angiotensin receptor blocker is eprosartan, olmesartan, valsartan, candesartan, losartan, telmisartan, irbesartan, valsartan, azilsartan medoxomil, or a combination of two or more thereof. In embodiments, the calcium channel blocker is amoldipine, aranidipine, azelnidipine, barnidipine, benidipine, cilnidine, clevidipine, efonidipine, felodipine, isradipine, lacidipine, lercanidipine, manidipine, nicardipine, nifedipine, nilvadipine, nimodipine, nisoldipine, nitrendipine, pranidipine, fendiline, gallipamil, verapamil, diltiazem, mibefradil, bepridil, flunarizine, fluspirilene, fendiline, gabapentin, pregabalin, ziconotide, or a combination of two or more thereof. When the subject is taking two or more hypertension medications, the hypertension medications are generally two or more different classes of hypertension medications.

In embodiments of the methods described herein, the low renin hypertensive subject or the low renin hypertensive subject has a plasma renin activity less than or equal to 1.0 units/milliliter/hour. In embodiments, the subject has a plasma renin activity less than or equal to 0.9 units/milliliter/hour. In embodiments, the subject has a plasma renin activity less than or equal to 0.8 units/milliliter/hour. In embodiments, the subject has a plasma renin activity less than or equal to 0.7 units/milliliter/hour. In embodiments, the subject has a plasma renin activity less than or equal to 0.6 units/milliliter/hour. In embodiments, the subject has a plasma renin activity less than or equal to 0.5 units/milliliter/hour. In embodiments, the plasma renin activity is taken as a 24-hour sample. In embodiments, plasma renin activity is taken during a blood test. In embodiments, plasma renin activity is taken during a urine test. Plasma renin activity can be measured by standard, commercially available tests known in the art. Such measurements can be conducted by FDA-approved laboratories. See, e.g., Hung et al, The Scientific World Journal, 2013: 294594 (2013). Many hypertension medications cause an increase in a subject's plasma renin activity. Accordingly, it will be understood by persons of skill in the art that a low renin hypertensive subject with a plasma renin activity less than or equal to 0.6 units/milliliter/hour when not taking a hypertension medication that increases plasma renin activity may also be a subject with a plasma renin activity less than or equal to 1 unit/milliliter/hour when taking one or more hypertension medications that increase plasma renin activity. It will also be understood by persons of skill in the art that an equivalent alternative to assays that measure plasma renin activity are assays that measure direct active renin (DAR) concentration. Accordingly, for any of the methods described herein that include a step of measuring plasma renin activity or identifying subjects with a plasma renin activity below a given threshold, there is alternative method that measures the subject's DAR concentration. For example, subjects that have a plasma renin activity of less than or equal to 0.6 units/milliliter/hour may be identified by measuring the subject's active renin concentration with an appropriate assay. A conversion rate of 1 ng/mL/h PRA to a DRA concentration of 8.4 mU/L is reported in Stowasser et al, Clin Biochem Rev, 31(2):39-56 (2010).

In embodiments of the methods described herein, the low renin hypertensive subject has a plasma aldosterone concentration of greater than or equal to 4 ng/dL. In embodiments, the subject has a plasma aldosterone concentration of greater than or equal to 5 ng/dL. In embodiments, the subject has a plasma aldosterone concentration of greater than or equal to 6 ng/dL. In embodiments, the subject has a plasma aldosterone concentration of greater than or equal to 7 ng/dL. Plasma aldosterone concentration can be measured by standard, commercially available tests known in the art. Such measurements can be conducted by FDA-approved laboratories. See, e.g., Stowasser et al, Clin Biochem Rev, 31(2):39-56 (2010), citing Schirpenbach, et al. Clinical chemistry 52, no. 9 (2006): 1749-1755. Notably, the assays for measuring aldosterone reported in Schirpenbach, et al. are immunoassays. As reported in Guo et al. The Journal of Clinical Endocrinology & Metabolism 103, no. 11 (2018): 3965-3973, LC-MS assays have been shown to have a higher specificity. As used herein, a plasma aldosterone concentration of greater than or equal to 6 ng/dL as measured by an immunoassay such as ELISA corresponds to a plasma aldosterone concentration of greater than or equal to about 1 ng/dL as measured by LC-MS.

In embodiments of the methods described herein, the low renin hypertensive subject or the low renin hypertensive subject has a systolic blood pressure of 120 mmHg or more. In embodiments, the subject has a systolic blood pressure of 130 mmHg or more. In embodiments, the subject has a systolic blood pressure of 140 mmHg or more. In embodiments, the subject has a systolic blood pressure greater than 140 mmHg. In embodiments, the subject has a systolic blood pressure of 150 mmHg or more. In embodiments, the subject has a systolic blood pressure of 160 mmHg or more. In embodiments of the methods described herein, the low renin hypertensive subject or the low renin hypertensive subject has a diastolic blood pressure of 70 mmHg or more. In embodiments, the subject has a diastolic blood pressure of 80 mmHg or more. In embodiments, the subject has a diastolic blood pressure of 90 mmHg or more. In embodiments, the subject has a diastolic blood pressure of greater than 90 mmHg. In embodiments, the subject has a diastolic blood pressure of 100 mmHg or more. In embodiments of the methods described herein, the low renin hypertensive subject or the low renin hypertensive subject has a systolic blood pressure of 120 mmHg or more and a diastolic blood pressure of 70 mmHg or more. In embodiments, the subject has a systolic blood pressure of 130 mmHg or more and a diastolic blood pressure of 80 mmHg or more. In embodiments, the subject has a systolic blood pressure of greater than 140 mmHg and a diastolic blood pressure of greater than 90 mmHg in the subject. Methods for measuring blood pressure are well-known in the art. In embodiments, blood pressure is measured by automated office blood pressure measurement (AOB). See Wright et al, N Engl J Med., 373(22):2103-2116 (2015) (correction published in N Engl J Med., 377(25):2506 (2017)). In embodiments, the automated office blood pressure measurement is taken by an automated oscillometric instrument. See, e.g., Andreadis et al, Journal of Clinical Hypertension, 22:555-559 (2020).

In embodiments, between 5 mg and 100 mg of the CYP 11β2 beta hydroxylase inhibitor is administered orally twice a day. In embodiments, between 10 mg and 50 mg of the CYP 11β2 beta hydroxylase inhibitor is administered orally twice a day, 12 hours apart. In embodiments, between 5 mg and 100 mg of the CYP 11β2 beta hydroxylase inhibitor is administered orally once a day. In embodiments, between 10 mg and 50 mg of the CYP 11β2 beta hydroxylase inhibitor is administered orally once a day.

In embodiments, 12.5 mg of the CYP 11β2 beta hydroxylase inhibitor is administered orally twice a day, 12 hours apart. In embodiments, 25 mg of the CYP 11β2 beta hydroxylase inhibitor is administered orally twice a day, 12 hours apart. In embodiments, 12.5 mg of the CYP 11β2 beta hydroxylase inhibitor is administered orally once a day. In embodiments, 50 mg of the CYP 11β2 beta hydroxylase inhibitor is administered orally once a day. In embodiments, 100 mg of the CYP 11β2 beta hydroxylase inhibitor is administered orally once a day.

In embodiments, the CYP 11β2 beta hydroxylase inhibitor does not inhibit the activity of 11β-hydroxylase in the subject as demonstrated by a lack of a clinically meaningful reduction in cortisol production in an ACTH (Cortrosyn) Stimulation test, preferably wherein the post-stimulation cortisol levels are greater 18 mcg/dL.

The ACTH stimulation test assesses the function of the adrenal glands and their ability to respond to ACTH. Adrenocorticotropic hormone is a hormone produced in the pituitary gland that stimulates the adrenal glands to release cortisol. Based on changes in cortisol levels, an ACTH stimulation test may be performed and the subject's dose of study drug may be withheld.

The ACTH stimulation test is recognized as the gold standard assay of adrenal insufficiency. ACTH stimulation tests may be performed in select patients based on low morning serum cortisol levels (ie, <3 mcg/dL) and clinical evidence of adrenal insufficiency. Cosyntropin is a synthetic form of ACTH. The test consists of the following procedures:
Obtain blood sample for pretest serum cortisol measurement (collected prior to administering synthetic ACTH)
Administer 0.25 mg synthetic ACTH via intravenous push
Obtain blood samples for serum cortisol measurements at 30 minutes and 60 minutes after dosing of synthetic ACTH

Post-stimulation serum cortisol levels should be greater than 18 mcg/dL.

In embodiments, the CYP 11β2 beta hydroxylase inhibitor is administered to the low renin hypertensive subject in an amount below the amount which causes an accumulation of 11-deoxycortisterone (11-DOC) above 0.1 ng/ml in the subject, and which:
(i) suppresses aldosterone production in the subject;
(ii) increases serum and/or plasma potassium levels in the subject; and/or
(iii) increases plasma renin activity (PRA) in the subject.

In embodiments, the CYP 11β2 beta hydroxylase inhibitor is administered to the low renin hypertensive subject in an amount which does not cause the subject's serum and/or plasma 11-DOC levels to exceed 600 pmol/L, In embodiments, the CYP 11β2 beta hydroxylase inhibitor is administered to the low renin hypertensive subject in an amount which does not cause the subject's serum and/or plasma 11-DOC levels to exceed 400 pmol/L.

In embodiments, serum and/or plasma aldosterone AUC-24 is reduced in the subject by at least 25% relative to serum and/or plasma aldosterone AUC-24 in the subject prior to administration of the CYP 11β2 beta hydroxylase inhibitor. In embodiments, serum and/or plasma potassium levels in the subject are increased by at least 0.3 mMol/L relative to the serum and/or plasma potassium levels in the subject prior to administration of the CYP 11β2 beta hydroxylase inhibitor. In embodiments, PRA in the subject is increased by at least 5 ng/nl/hr relative to the PRA in the subject prior to administration of the CYP 11β2 beta hydroxylase inhibitor.

In embodiments, the CYP 11β2 beta hydroxylase inhibitor is administered to the low renin hypertensive subject in an amount which does not cause a clinically meaningful upregulation of the subject's adrenocortical hormone synthesis.

In embodiments, the administration of the CYP 11β2 beta hydroxylase inhibitor is administered to the low renin hypertensive subject in an amount which:
(i) does not cause a clinically meaningful reduction of the subject's serum and/or plasma cortisol levels, relative to the subject's serum and/or plasma cortisol levels prior to administration of the CYP 11β2 beta hydroxylase inhibitor;
(ii) does not cause a clinically meaningful increase in the subject's serum and/or plasma 11-DOC levels relative to the subject's serum and/or plasma 11-DOC levels prior to administration of the CYP 11β2 beta hydroxylase inhibitor; and/or
(iii) does not cause a clinically meaningful increase in the subject's serum and/or plasma 11-deoxycortisol levels relative to the subject's serum and/or plasma 11-deoxycortisol levels prior to administration of the CYP 11β2 beta hydroxylase inhibitor.

In embodiments, the CYP 11β2 beta hydroxylase inhibitor is administered to the low renin hypertensive subject in an amount:
(i) which does not cause a reduction of more than 20% in the subject's serum and/or plasma cortisol levels, relative to the subject's serum and/or plasma cortisol levels prior to administration of the CYP 11β2 hydroxylase inhibitor, preferably which does not cause a reduction of more than 10% in the subject's serum and/or plasma cortisol levels, relative to the subject's serum and/or plasma cortisol levels prior to administration of the CYP 11β2 beta hydroxylase inhibitor;
(ii) which does not cause an increase of more than 20% in the subject's serum and/or plasma 11-DOC levels relative to the subject's serum and/or plasma 11-DOC levels prior to administration of the CYP 11β2 beta hydroxylase inhibitor, preferably which does not cause an increase of more than 10% in the subject's serum and/or plasma 11-DOC levels relative to the subject's serum and/or plasma 11-DOC levels prior to administration of the CYP 11β2 beta hydroxylase inhibitor; and/or
(iii) which does not cause an increase of more than 20% in the subject's serum and/or plasma 11-deoxycortisol levels relative to the subject's serum and/or plasma 11-deoxycortisol levels prior to administration of the CYP 11β2 beta hydroxylase inhibitor, preferably which does not cause an increase of more than 10% in the subject's serum and/or plasma 11-deoxycortisol levels relative to the subject's serum and/or plasma 11-deoxycortisol levels prior to administration of the CYP 11β2 beta hydroxylase inhibitor.

In an embodiment, the subject's office-measured systolic blood pressure is lowered relative to the subject's office-measured systolic blood pressure prior to administration of the CYP 11β2 beta hydroxylase inhibitor. In an embodiment, the subject's ambulatory systolic blood pressure is lowered relative to the subject's ambulatory systolic blood pressure prior to administration of the CYP 11β2 beta hydroxylase inhibitor. In embodiments, the subject's ambulatory systolic blood pressure is measured by automated office blood pressure measurement (AOB) or sphygmomanometer.

In an embodiment, the subject's office-measured systolic blood pressure is lowered by at least 10 mmHg relative to the subject's office-measured systolic blood pressure prior to administration of the CYP 11β2 beta hydroxylase inhibitor. In an embodiment, the subject's ambulatory systolic blood pressure is lowered by at least 10 mmHg relative to the subject's ambulatory systolic blood pressure prior to administration of the CYP 11β2 beta hydroxylase inhibitor.

In an embodiment, the subject's ambulatory systolic and diastolic blood pressure is lowered relative to the subject's ambulatory systolic and diastolic blood pressure prior to administration of the CYP 11β2 beta hydroxylase inhibitor. In an embodiment, the subject's office-measured systolic and diastolic blood pressure is lowered relative to the subject's office-measured systolic and diastolic blood pressure prior to administration of the CYP 11β2 beta hydroxylase inhibitor. In an embodiment, the subject's office-measured diastolic blood pressure is lowered relative to the subject's office-measured diastolic blood pressure prior to administration of the CYP 11β2 beta hydroxylase inhibitor. In an embodiment, the subject's systolic blood pressure is reduced to less than 130 mmHg and/or the subject's diastolic blood pressure is reduced to less than 80 mmHg.

In an embodiment, the subject's ambulatory systolic blood pressure is lowered by at least 5 mmHg, preferably by at least 10 mmHg and the subject's ambulatory diastolic blood pressure is lowered by at least 2.5 mmHg, preferably by at least 5 mmHg, each relative to the subject's ambulatory systolic and diastolic blood pressure, respectively, prior to administration of the CYP 11β2 beta hydroxylase inhibitor. In an embodiment, the subject's office-measured systolic blood pressure is lowered by at least 5 mmHg, preferably by at least 10 mmHg and the subject's office-measured diastolic blood pressure is lowered by at least 2.5 mmHg, preferably by at least 5 mmHg, each relative to the subject's office-measured systolic and diastolic blood pressure, respectively, prior to administration of the CYP 11β2 beta hydroxylase inhibitor. In an embodiment, the subject's office-measured diastolic blood pressure is lowered by at least 2.5 mmHg, preferably by at least 5 mmHg relative to the subject's office-measured diastolic blood pressure prior to administration of the CYP 11β2 beta hydroxylase inhibitor. In an embodiment, the subject's systolic blood pressure is reduced to less than 130 mmHg and/or the subject's diastolic blood pressure is reduced to less than 80 mmHg.

In an embodiment, the subject's ambulatory systolic blood pressure is lowered by between 2.5 and 10 mmHg, preferably by between 5 and 15 mmHg and the subject's ambulatory diastolic blood pressure is lowered by between 2.5 and 7.5 mmHg, preferably by between 5 and 10 mmHg, each relative to the subject's ambulatory systolic and diastolic blood pressure, respectively, prior to administration of the CYP 11β2 beta hydroxylase inhibitor. In an embodiment, the subject's office-measured systolic blood pressure is lowered by between 2.5 and 10 mmHg, preferably by between 5 and 15 mmHg and the subject's office-measured diastolic blood pressure is lowered by between 2.5 and 7.5 mmHg, preferably by between 5 and 10 mmHg, each relative to the subject's office-measured systolic and diastolic blood pressure, respectively, prior to administration of the CYP 11β2 beta hydroxylase inhibitor. In an embodiment, the subject's office-measured diastolic blood pressure is lowered by between 2.5 and 7.5 mmHg, preferably by between 5 and 10 mmHg relative to the subject's office-measured diastolic blood pressure prior to administration of the CYP 11β2 beta hydroxylase inhibitor. In an embodiment, the subject's systolic blood pressure is reduced to less than 130 mmHg and/or the subject's diastolic blood pressure is reduced to less than 80 mmHg.

All combinations of the various elements disclosed above are within the scope of the invention. The following are non-limiting examples of such embodiments.

In an embodiment, 12.5 mg of the CYP 11β2 beta hydroxylase inhibitor, preferably the CYP 11β2 beta hydroxylase inhibitor of Formula (A), is administered twice a day, 12 hours apart and one or more or all of the following features apply: (a) the CYP 11β2 beta hydroxylase inhibitor does not inhibit the activity of 11β-hydroxylase in the subject as demonstrated by a lack of a clinically meaningful reduction in cortisol production in an ACTH (Cortrosyn) Stimulation test, preferably wherein the post-stimulation cortisol levels are greater 18 mcg/d; (b) the CYP 11β2 beta hydroxylase does not cause an accumulation of 11-deoxycortisterone (11-DOC) above 0.1 ng/ml in the subject, and suppresses aldosterone production in the subject, increases serum and/or plasma potassium levels in the subject, and/or increases plasma renin activity (PRA) in the subject; (c) the CYP 11β2 beta hydroxylase inhibitor does not cause a clinically meaningful upregulation of the subject's adrenocortical hormone synthesis; (d) the CYP 11β2 beta hydroxylase inhibitor does not cause a clinically meaningful reduction of the subject's serum and/or plasma cortisol levels, relative to the subject's serum and/or plasma cortisol levels prior to administration of the CYP 11β2 beta hydroxylase; (e) the CYP 11β2 beta hydroxylase inhibitor does not cause a clinically meaningful increase in the subject's serum and/or plasma 11-DOC levels relative to the subject's serum and/or plasma 11-DOC levels prior to administration of the CYP 11β2 beta hydroxylase inhibitor; (f) the CYP 11β2 beta hydroxylase inhibitor does not cause a clinically meaningful increase in the subject's serum and/or plasma 11-deoxycortisol levels relative to the subject's serum and/or plasma 11-deoxycortisol levels prior to administration of the CYP 11β2 beta hydroxylase inhibitor; (g) the subject's office-measured systolic and/or diastolic blood pressure is lowered relative to the subject's office-measured systolic blood pressure prior to administration of the CYP 11β2 beta hydroxylase inhibitor; (h) the subject's ambulatory systolic and/or diastolic blood pressure is lowered relative to the subject's ambulatory systolic blood pressure prior to administration of the CYP 11β2 beta hydroxylase inhibitor. All increases or decreases (as applicable) described herein for levels of cortisol, 11-DOC, 11-deoxycortisol, aldosterone, potassium, PRA, blood pressure etc. are part of this embodiment.

In an embodiment, 25 mg of the CYP 11β2 beta hydroxylase inhibitor, preferably the CYP 11β2 beta hydroxylase inhibitor of Formula (A), is administered twice a day, 12 hours apart and one or more or all of the following features apply: (a) the CYP 11β2 beta hydroxylase inhibitor does not inhibit the activity of 11β-hydroxylase in the subject as demonstrated by a lack of a clinically meaningful reduction in cortisol production in an ACTH (Cortrosyn) Stimulation test, preferably wherein the post-stimulation cortisol levels are greater 18 mcg/d; (b) the CYP 11β2 beta hydroxylase does not cause an accumulation of 11-deoxycortisterone (11-DOC) above 0.1 ng/ml in the subject, and suppresses aldosterone production in the subject, increases serum and/or plasma potassium levels in the subject, and/or increases plasma renin activity (PRA) in the subject; (c) the CYP 11β2 beta hydroxylase inhibitor does not cause a clinically meaningful upregulation of the subject's adrenocortical hormone synthesis; (d) the CYP 11β2 beta hydroxylase inhibitor does not cause a clinically meaningful reduction of the subject's serum and/or plasma cortisol levels, relative to the subject's serum and/or plasma cortisol levels prior to administration of the CYP 11β2 beta hydroxylase; (e) the CYP 11β2 beta hydroxylase inhibitor does not cause a clinically meaningful increase in the subject's serum and/or plasma 11-DOC levels relative to the subject's serum and/or plasma 11-DOC levels prior to administration of the CYP 11β2 beta hydroxylase inhibitor; (f) the CYP 11β2 beta hydroxylase inhibitor does not cause a clinically meaningful increase in the subject's serum and/or plasma 11-deoxycortisol levels relative to the subject's serum and/or plasma 11-deoxycortisol levels prior to administration of the CYP 11β2 beta hydroxylase inhibitor; (g) the subject's office-measured systolic and/or diastolic blood pressure is lowered relative to the subject's office-measured systolic blood pressure prior to administration of the CYP 11β2 beta hydroxylase inhibitor; (h) the subject's ambulatory systolic and/or diastolic blood pressure is lowered relative to the subject's ambulatory systolic blood pressure prior to administration of the CYP 11β2 beta hydroxylase inhibitor. All increases or decreases (as applicable) described herein for levels of cortisol, 11-DOC, 11-deoxycortisol, aldosterone, potassium, PRA, blood pressure etc. are part of this embodiment.

In an embodiment, 12.5 mg of the CYP 11β2 beta hydroxylase inhibitor, preferably the CYP 11β2 beta hydroxylase inhibitor of Formula (A), is administered once a day and one or more or all of the following features apply: (a) the CYP 11β2 beta hydroxylase inhibitor does not inhibit the activity of 11β-hydroxylase in the subject as demonstrated by a lack of a clinically meaningful reduction in cortisol production in an ACTH (Cortrosyn) Stimulation test, preferably wherein the post-stimulation cortisol levels are greater 18 mcg/d; (b) the CYP 11β2 beta hydroxylase does not cause an accumulation of 11-deoxycortisterone (11-DOC) above 0.1 ng/ml in the subject, and suppresses aldosterone production in the subject, increases serum and/or plasma potassium levels in the subject, and/or increases plasma renin activity (PRA) in the subject; (c) the CYP 11β2 beta hydroxylase inhibitor does not cause a clinically meaningful upregulation of the subject's adrenocortical hormone synthesis; (d) the CYP 11β2 beta hydroxylase inhibitor does not cause a clinically meaningful reduction of the subject's serum and/or plasma cortisol levels, relative to the subject's serum and/or plasma cortisol levels prior to administration of the CYP 11β2 beta hydroxylase; (e) the CYP 11β2 beta hydroxylase inhibitor does not cause a clinically meaningful increase in the subject's serum and/or plasma 11-DOC levels relative to the subject's serum and/or plasma 11-DOC levels prior to administration of the CYP 11β2 beta hydroxylase inhibitor; (f) the CYP 11β2 beta hydroxylase inhibitor does not cause a clinically meaningful increase in the subject's serum and/or plasma 11-deoxycortisol levels relative to the subject's serum and/or plasma 11-deoxycortisol levels prior to administration of the CYP 11β2 beta hydroxylase inhibitor; (g) the subject's office-measured systolic and/or diastolic blood pressure is lowered relative to the subject's office-measured systolic blood pressure prior to administration of the CYP 11β2 beta hydroxylase inhibitor; (h) the subject's ambulatory systolic and/or diastolic blood pressure is lowered relative to the subject's ambulatory systolic blood pressure prior to administration of the CYP 11β2 beta hydroxylase inhibitor. All increases or decreases (as applicable) described herein for levels of cortisol, 11-DOC, 11-deoxycortisol, aldosterone, potassium, PRA, blood pressure etc. are part of this embodiment.

In an embodiment, 25 mg of the CYP 11β2 beta hydroxylase inhibitor, preferably the CYP 11β2 beta hydroxylase inhibitor of Formula (A), is administered once a day and one or more or all of the following features apply: (a) the CYP 11β2 beta hydroxylase inhibitor does not inhibit the activity of 11β-hydroxylase in the subject as demonstrated by a lack of a clinically meaningful reduction in cortisol production in an ACTH (Cortrosyn) Stimulation test, preferably wherein the post-stimulation cortisol levels are greater 18 mcg/d; (b) the CYP 11β2 beta hydroxylase does not cause an accumulation of 11-deoxycortisterone (11-DOC) above 0.1 ng/ml in the subject, and suppresses aldosterone production in the subject, increases serum and/or plasma potassium levels in the subject, and/or increases plasma renin activity (PRA) in the subject; (c) the CYP 11β2 beta hydroxylase inhibitor does not cause a clinically meaningful upregulation of the subject's adrenocortical hormone synthesis; (d) the CYP 11β2 beta hydroxylase inhibitor does not cause a clinically meaningful reduction of the subject's serum and/or plasma cortisol levels, relative to the subject's serum and/or plasma cortisol levels prior to administration of the CYP 11β2 beta hydroxylase; (e) the CYP 11β2 beta hydroxylase inhibitor does not cause a clinically meaningful increase in the subject's serum and/or plasma 11-DOC levels relative to the subject's serum and/or plasma 11-DOC levels prior to administration of the CYP 11β2 beta hydroxylase inhibitor; (f) the CYP 11β2 beta hydroxylase inhibitor does not cause a clinically meaningful increase in the subject's serum and/or plasma 11-deoxycortisol levels relative to the subject's serum and/or plasma 11-deoxycortisol levels prior to administration of the CYP 11β2 beta hydroxylase inhibitor; (g) the subject's office-measured systolic and/or diastolic blood pressure is lowered relative to the subject's office-measured systolic blood pressure prior to administration of the CYP 11β2 beta hydroxylase inhibitor; (h) the subject's ambulatory systolic and/or diastolic blood pressure is lowered relative to the subject's ambulatory systolic blood pressure prior to administration of the CYP 11β2 beta hydroxylase inhibitor. All increases or decreases (as applicable) described herein for levels of cortisol, 11-DOC, 11-deoxycortisol, aldosterone, potassium, PRA, blood pressure etc. are part of this embodiment.

In an embodiment, 50 mg of the CYP 11β2 beta hydroxylase inhibitor, preferably the CYP 11β2 beta hydroxylase inhibitor of Formula (A), is administered once a day and one or more or all of the following features apply: (a) the CYP 11β2 beta hydroxylase inhibitor does not inhibit the activity of 11β-hydroxylase in the subject as demonstrated by a lack of a clinically meaningful reduction in cortisol production in an ACTH (Cortrosyn) Stimulation test, preferably wherein the post-stimulation cortisol levels are greater 18 mcg/d; (b) the CYP 11β2 beta hydroxylase does not cause an accumulation of 11-deoxycortisterone (11-DOC) above 0.1 ng/ml in the subject, and suppresses aldosterone production in the subject, increases serum and/or plasma potassium levels in the subject, and/or increases plasma renin activity (PRA) in the subject; (c) the CYP 11β2 beta hydroxylase inhibitor does not cause a clinically meaningful upregulation of the subject's adrenocortical hormone synthesis; (d) the CYP 11β2 beta hydroxylase inhibitor does not cause a clinically meaningful reduction of the subject's serum and/or plasma cortisol levels, relative to the subject's serum and/or plasma cortisol levels prior to administration of the CYP 11β2 beta hydroxylase; (e) the CYP 11β2 beta hydroxylase inhibitor does not cause a clinically meaningful increase in the subject's serum and/or plasma 11-DOC levels relative to the subject's serum and/or plasma 11-DOC levels prior to administration of the CYP 11β2 beta hydroxylase inhibitor; (f) the CYP 11β2 beta hydroxylase inhibitor does not cause a clinically meaningful increase in the subject's serum and/or plasma 11-deoxycortisol levels relative to the subject's serum and/or plasma 11-deoxycortisol levels prior to administration of the CYP 11β2 beta hydroxylase inhibitor; (g) the subject's office-measured systolic and/or diastolic blood pressure is lowered relative to the subject's office-measured systolic blood pressure prior to administration of the CYP 11β2 beta hydroxylase inhibitor; (h) the subject's ambulatory systolic and/or diastolic blood pressure is lowered relative to the subject's ambulatory systolic blood pressure prior to administration of the CYP 11β2 beta hydroxylase inhibitor. All increases or decreases (as applicable) described herein for levels of cortisol, 11-DOC, 11-deoxycortisol, aldosterone, potassium, PRA, blood pressure etc. are part of this embodiment.

In an embodiment, 100 mg of the CYP 11β2 beta hydroxylase inhibitor, preferably the CYP 11β2 beta hydroxylase inhibitor of Formula (A), is administered once a day and one or more or all of the following features apply: (a) the CYP 11β2 beta hydroxylase inhibitor does not inhibit the activity of 11β-hydroxylase in the subject as demonstrated by a lack of a clinically meaningful reduction in cortisol production in an ACTH (Cortrosyn) Stimulation test, preferably wherein the post-stimulation cortisol levels are greater 18 mcg/d; (b) the CYP 11β2 beta hydroxylase does not cause an accumulation of 11-deoxycortisterone (11-DOC) above 0.1 ng/ml in the subject, and suppresses aldosterone production in the subject, increases serum and/or plasma potassium levels in the subject, and/or increases plasma renin activity (PRA) in the subject; (c) the CYP 11β2 beta hydroxylase inhibitor does not cause a clinically meaningful upregulation of the subject's adrenocortical hormone synthesis; (d) the CYP 11β2 beta hydroxylase inhibitor does not cause a clinically meaningful reduction of the subject's serum and/or plasma cortisol levels, relative to the subject's serum and/or plasma cortisol levels prior to administration of the CYP 11β2 beta hydroxylase; (e) the CYP 11β2 beta hydroxylase inhibitor does not cause a clinically meaningful increase in the subject's serum and/or plasma 11-DOC levels relative to the subject's serum and/or plasma 11-DOC levels prior to administration of the CYP 11β2 beta hydroxylase inhibitor; (f) the CYP 11β2 beta hydroxylase inhibitor does not cause a clinically meaningful increase in the subject's serum and/or plasma 11-deoxycortisol levels relative to the subject's serum and/or plasma 11-deoxycortisol levels prior to administration of the CYP 11β2 beta hydroxylase inhibitor; (g) the subject's office-measured systolic and/or diastolic blood pressure is lowered relative to the subject's office-measured systolic blood pressure prior to administration of the CYP 11β2 beta hydroxylase inhibitor; (h) the subject's ambulatory systolic and/or diastolic blood pressure is lowered relative to the subject's ambulatory systolic blood pressure prior to administration of the CYP 11β2 beta hydroxylase inhibitor. All increases or decreases (as applicable) described herein for levels of cortisol, 11-DOC, 11-deoxycortisol, aldosterone, potassium, PRA, blood pressure etc. are part of this embodiment.

Also provided herein are any of CYP 11β2 beta hydroxylase inhibitors or compositions described herein for use in treating a low renin hypertensive subject.

In embodiments, said low renin hypertensive subject is taking or has taken a hypertension medication selected from a diuretic, an ACE inhibitor, an angiotensin receptor blocker, a calcium channel blocker, or a combination of two or more thereof. In embodiments, the low renin hypertensive subject is taking or has taken at least two of said hypertension medications. In embodiments, the low renin hypertensive subject has a plasma renin activity less than or equal to 0.6 units/milliliter/hour. In embodiments, the low renin hypertensive subject has a plasma aldosterone concentration of greater than or equal to 6 ng/dL. Any combination of these features of the low renin hypertensive subject is within the scope of the invention.

In an embodiment, the CYP 11β2 beta hydroxylase inhibitors and compositions described herein for use in treating a low renin hypertensive subject which, when administered to a low renin hypertensive subject, will cause one or more or all of the effects on the subject described in paragraphs to.

Methods of Identification, Selection, or Treatment of a Subject

The disclosure provides methods of identifying a subject for hypertension treatment with a CYP 11β2 beta hydroxylase inhibitor, the method comprising: (i) measuring a systolic blood pressure of greater than 140 mmHg in the subject; (ii) measuring a diastolic blood pressure of greater than 90 mmHg in the subject; (iii) measuring a plasma renin activity less than or equal to 0.6 units/milliliter/hour in the subject; (iv) measuring a plasma aldosterone concentration of greater than or equal to 6 ng/dL in the subject; or (v) measuring a combination of two or more of the foregoing; thereby identifying the subject for hypertension treatment with a CYP 11β2 beta hydroxylase inhibitor. The disclosure provides methods of identifying a subject for hypertension treatment with a CYP 11β2 beta hydroxylase inhibitor, the method comprising: (i) measuring a systolic blood pressure of greater than 130 mmHg in the subject; (ii) measuring a diastolic blood pressure of greater than 90 mmHg in the subject; (iii) determining that the subject has a plasma renin activity less than or equal to 0.6 units/milliliter/hour when not taking a hypertension medication or a plasma renin activity less than or equal to 1 unit/milliliter/hour when taking one or more hypertension medications; (iv) determining that the subject has a plasma aldosterone concentration of greater than or equal to 6 ng/dL if measured by an immunoassay such as ELISA or greater than or equal to 1 ng/dL if measured by LC-MS; or (v) determining a combination of two or more of the foregoing; thereby identifying the subject for hypertension treatment with a CYP 11β2 beta hydroxylase inhibitor. In embodiments, the subject is a low renin hypertensive subject or a low renin hypertensive subject. In embodiments, the CYP11β2 beta hydroxylase inhibitor is a compound described in U.S. Pat. No. 10,029,993, the disclosure of which is incorporated by reference herein. In embodiments, the CYP11β2 beta hydroxylase inhibitor is a compound described in U.S. Pat. No. 10,329,263, the disclosure of which is incorporated by reference herein. In embodiments, the CYP 11β2 beta hydroxylase inhibitor is a 1,2,4-triazine compound or a pharmaceutically acceptable salt thereof. In embodiments, the CYP11β2 beta hydroxylase inhibitor is a compound of Formula (A) or a pharmaceutically acceptable salt thereof:

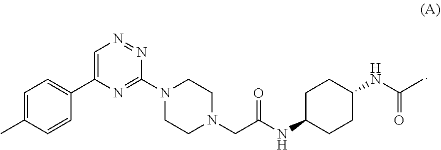 [SMILES: CC(=O)N[C@H]1CC[C@H](NC(=O)CN2CCN(c3nncc(-c4ccc(C)cc4)n3)CC2)CC1]

In embodiments, the CYP11β2 beta hydroxylase inhibitor is a pharmaceutically acceptable salt of the compound of Formula (A). In embodiments, the CYP11β2 beta hydroxylase inhibitor is a monohydrobromide salt of the compound of Formula (A). In embodiments, the CYP11β2 beta hydroxylase inhibitor is an antibody (e.g., antibody inhibitor). In embodiments, the CYP11β2 beta hydroxylase inhibitor is an antisense nucleic acid (e.g., antisense nucleic acid inhibitor). In embodiments, the CYP11β2 beta hydroxylase inhibitor is an aptamer (e.g., aptamer inhibitor). In embodiments, the CYP11β2 beta hydroxylase inhibitor is a small molecule (e.g., small molecule inhibitor). In embodiments, the CYP11β2 beta hydroxylase inhibitor is a natural ligand, a protein inhibitor, a biomolecule inhibitor, or the like. In embodiments, the methods comprise measuring at least two of (i), (ii), (iii), and (iv). In embodiments, the methods comprise measuring at least three of (i), (ii), (iii), and (iv). In embodiments, the methods comprise measuring (i), (ii), (iii), and (iv). In embodiments, the methods comprise measuring (i) and (ii). In embodiments, the methods comprise measuring (i) and (iii). In embodiments, the methods comprise measuring (i) and (iv). In embodiments, the methods comprise measuring (ii) and (iii). In embodiments, the methods comprise measuring (ii) and (iv). In embodiments, the methods comprise measuring (iii) and (iv). In embodiments, the methods comprise measuring (i), (ii), and (iii). In embodiments, the methods comprise measuring (i), (ii), and (iv). In embodiments, the methods comprise measuring (i), (iii), and (iv). In embodiments, the methods comprise measuring (ii), (iii), and (iv). In embodiments, the methods comprise determining at least two of (i), (ii), (iii), and (iv). In embodiments, the methods comprise determining at least three of (i), (ii), (iii), and (iv). In embodiments, the methods comprise determining (i), (ii), (iii), and (iv). In embodiments, the methods comprise determining (i) and (ii). In embodiments, the methods comprise determining (i) and (iii). In embodiments, the methods comprise determining (i) and (iv). In embodiments, the methods comprise determining (ii) and (iii). In embodiments, the methods comprise determining (ii) and (iv). In embodiments, the methods comprise determining (iii) and (iv). In embodiments, the methods comprise determining (i), (ii), and (iii). In embodiments, the methods comprise determining (i), (ii), and (iv). In embodiments, the methods comprise determining (i), (iii), and (iv). In embodiments, the methods comprise determining (ii), (iii), and (iv).

The disclosure provides methods of treating hypertension in a subject in need thereof comprising: (a) measuring in the subject, or determining that the subject has: (i) a systolic blood pressure of greater than 140 mmHg; (ii) a diastolic blood pressure of greater than 90 mmHg; (iii) a plasma renin activity less than or equal to 0.6 units/milliliter/hour when not taking a hypertension medication or a plasma renin activity less than or equal to 1 units/milliliter/hour when taking a hypertension medication; (iv) a plasma aldosterone concentration of greater than or equal to 6 ng/dL if measured by an immunoassay such as ELISA or greater than or equal to 1 ng/dL if measured by LC-MS; or (v) a combination of two or more of the foregoing; and (b) administering to the subject an effective amount of a CYP 11β2 beta hydroxylase inhibitor. In embodiments, the subject is a low renin hypertensive subject or a low renin hypertensive subject. In embodiments, the CYP11β2 beta hydroxylase inhibitor is a compound described in U.S. Pat. No. 10,029,993, the disclosure of which is incorporated by reference herein. In embodiments, the CYP11β2 beta hydroxylase inhibitor is a compound described in U.S. Pat. No. 10,329,263, the disclosure of which is incorporated by reference herein. In embodiments, the CYP 11β2 beta hydroxylase inhibitor is a 1,2,4-triazine compound or a pharmaceutically acceptable salt thereof. In embodiments, the CYP11β2 beta hydroxylase inhibitor is a compound of Formula (A) or a pharmaceutically acceptable salt thereof:

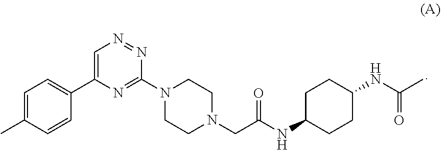 [SMILES: CC(=O)N[C@H]1CC[C@H](NC(=O)CN2CCN(c3nncc(-c4ccc(C)cc4)n3)CC2)CC1]

In embodiments, the CYP11β2 beta hydroxylase inhibitor is a pharmaceutically acceptable salt of the compound of Formula (A). In embodiments, the CYP11β2 beta hydroxylase inhibitor is a monohydrobromide salt of the compound of Formula (A). In embodiments, the CYP11β2 beta hydroxylase inhibitor is an antibody (e.g., antibody inhibitor). In embodiments, the CYP11β2 beta hydroxylase inhibitor is an antisense nucleic acid (e.g., antisense nucleic acid inhibitor). In embodiments, the CYP11β2 beta hydroxylase inhibitor is an aptamer (e.g., aptamer inhibitor). In embodiments, the CYP11β2 beta hydroxylase inhibitor is a small molecule (e.g., small molecule inhibitor). In embodiments, the CYP11β2 beta hydroxylase inhibitor is a natural ligand, a protein inhibitor, a biomolecule inhibitor, or the like. In embodiments, the methods comprise measuring at least two of (i), (ii), (iii), and (iv). In embodiments, the methods comprise measuring or determining at least three of (i), (ii), (iii), and (iv). In embodiments, the methods comprise measuring or determining (i), (ii), (iii), and (iv). In embodiments, the methods comprise measuring or determining (i) and (ii). In embodiments, the methods comprise measuring or determining (i) and (iii). In embodiments, the methods comprise measuring or determining (i) and (iv). In embodiments, the methods comprise measuring or determining (ii) and (iii). In embodiments, the methods comprise measuring or determining (ii) and (iv). In embodiments, the methods comprise measuring or determining (iii) and (iv). In embodiments, the methods comprise measuring or determining (i), (ii), and (iii). In embodiments, the methods comprise measuring or determining (i), (ii), and (iv). In embodiments, the methods comprise measuring (i), (iii), and (iv). In embodiments, the methods comprise measuring or determining (ii), (iii), and (iv).

In embodiments, the methods further comprise identifying that the subject is taking or has taken a hypertension medication. In embodiments, the methods further comprise identifying that the subject is taking a hypertension medication. In embodiments, the methods further comprise identifying that the subject is taking or has taken two hypertension medications. In embodiments, the methods further comprise identifying that the subject is taking two hypertension medications. In embodiments, the methods further comprise identifying that the subject is taking or has taken three or more hypertension medications. In embodiments, the methods further comprise identifying that the subject is taking three or more hypertension medications. In embodiments, the methods further comprise identifying that the subject is taking at least one hypertension medication selected from the group consisting of a diuretic, an ACE inhibitor, an angiotensin receptor blocker, a calcium channel blocker, or a combination of two or more thereof. In embodiments, the methods further comprise identifying that the subject is taking at least two hypertension medications selected from the group consisting of a diuretic, an ACE inhibitor, an angiotensin receptor blocker, a calcium channel blocker, or a combination of two or more thereof. In embodiments, the methods further comprise identifying that the subject is taking two hypertension medications selected from the group consisting of a diuretic, an ACE inhibitor, an angiotensin receptor blocker, a calcium channel blocker, or a combination of two or more thereof.

In embodiments of the methods described herein, the methods comprise measuring a plasma renin activity less than or equal to 1.0 units/milliliter/hour in the subject. In embodiments, the methods comprise measuring a plasma renin activity less than or equal to 0.9 units/milliliter/hour in the subject. In embodiments, the methods comprise measuring a plasma renin activity less than or equal to 0.8 units/milliliter/hour in the subject. In embodiments, the methods comprise measuring a plasma renin activity less than or equal to 0.7 units/milliliter/hour in the subject. In embodiments, the methods comprise measuring a plasma renin activity less than or equal to 0.6 units/milliliter/hour in the subject. In embodiments, the methods comprise measuring a plasma renin activity less than or equal to 0.5 units/milliliter/hour in the subject. In embodiments, the plasma renin activity is taken as a 24-hour sample. In embodiments, plasma renin activity is taken during a blood test. In embodiments, plasma renin activity is taken during a urine test. Plasma renin activity can be measured by standard, commercially available tests known in the art. Such measurements can be conducted by FDA-approved laboratories. See, e.g., Hung et al, The Scientific World Journal, 2013: 294594 (2013). Many hypertension medications cause an increase in a subject's plasma renin activity. Accordingly, it will be understood by persons of skill in the art that a low renin hypertensive subject with a plasma renin activity less than or equal to 0.6 units/milliliter/hour when not taking a hypertension medication that increases plasma renin activity may also be a subject with a plasma renin activity less than or equal to 1 unit/milliliter/hour when taking one or more hypertension medications that increase plasma renin activity. It will also be understood by persons of skill in the art that an equivalent alternative to assays that measure plasma renin activity are assays that measure direct active renin (DAR) concentration. Accordingly, for any of the methods described herein that include a step of measuring plasma renin activity or identifying subjects with a plasma renin activity below a given threshold, there is alternative method that measures the subject's DAR concentration. For example, subjects that have a plasma renin activity of less than or equal to 0.6 units/milliliter/hour may be identified by measuring the subject's active renin concentration with an appropriate assay. A conversion rate of 1 ng/mL/h PRA to a DRA concentration of 8.4 mU/L is reported in Stowasser et al, Clin Biochem Rev, 31(2):39-56 (2010).

In embodiments of the methods described herein, the methods comprise measuring a plasma aldosterone concentration of greater than or equal to 4 ng/dL in the subject. In embodiments, the methods comprise measuring a plasma aldosterone concentration of greater than or equal to 5 ng/dL in the subject. In embodiments, the methods comprise measuring a plasma aldosterone concentration of greater than or equal to 6 ng/dL in the subject. In embodiments, the methods comprise measuring a plasma aldosterone concentration of greater than or equal to 7 ng/dL in the subject. Plasma aldosterone concentration can be measured by standard, commercially available tests known in the art. Such measurements can be conducted by FDA-approved laboratories. See, e.g., Stowasser et al, Clin Biochem Rev, 31(2):39-56 (2010), citing Schirpenbach, et al. Clinical chemistry 52, no. 9 (2006): 1749-1755. Notably, the assays for measuring aldosterone reported in Schirpenbach, et al. are immunoassays. As reported in Guo et al. The Journal of Clinical Endocrinology & Metabolism 103, no. 11 (2018): 3965-3973, LC-MS assays have been shown to have a higher specificity, meaning that a plasma aldosterone concentration of greater than or equal to 6 ng/dL as measured by an immunoassay such as ELISA corresponds to a plasma aldosterone concentration of greater than or equal to about 1 ng/dL as measured by LC-MS.

In embodiments of the methods described herein, the methods comprise measuring a systolic blood pressure of 120 mmHg or more in the subject. In embodiments, the methods comprise measuring a systolic blood pressure of 130 mmHg or more in the subject. In embodiments, the methods comprise measuring a systolic blood pressure of 140 mmHg or more in the subject. In embodiments, the methods comprise measuring a systolic blood pressure greater than 140 mmHg in the subject. In embodiments, the methods comprise measuring a systolic blood pressure of 150 mmHg or more in the subject. In embodiments, the methods comprise measuring a systolic blood pressure of 160 mmHg or more in the subject. In embodiments, the methods comprise measuring has a diastolic blood pressure of 70 mmHg or more in the subject. In embodiments, the methods comprise measuring a diastolic blood pressure of 80 mmHg or more in the subject. In embodiments, the methods comprise measuring a diastolic blood pressure of 90 mmHg or more in the subject. In embodiments, the methods comprise measuring a diastolic blood pressure of greater than 90 mmHg in the subject. In embodiments, the methods comprise measuring a diastolic blood pressure of 100 mmHg or more in the subject. In embodiments, the methods comprise measuring a systolic blood pressure of 120 mmHg or more and a diastolic blood pressure of 70 mmHg or more in the subject. In embodiments, the methods comprise measuring a systolic blood pressure of 130 mmHg or more and a diastolic blood pressure of 80 mmHg or more in the subject. In embodiments, the methods comprise measuring a systolic blood pressure of greater than 140 mmHg and a diastolic blood pressure of greater than 90 mmHg in the subject. Methods for measuring blood pressure are well-known in the art. In embodiments, blood pressure is measured by automated office blood pressure measurement (AOB). See Wright et al, N Engl J Med., 373(22):2103-2116 (2015) (correction published in N Engl J Med., 377(25):2506 (2017)). In embodiments, the automated office blood pressure measurement is taken by an automated oscillometric instrument. See, e.g., Andreadis et al, Journal of Clinical Hypertension, 22:555-559 (2020).

Pharmaceutical Compositions

The disclosure provides pharmaceutical compositions comprising a CYP11β2 beta hydroxylase inhibitor and a pharmaceutically acceptable excipient. In embodiments, the CYP11β2 beta hydroxylase inhibitor is a compound described in U.S. Pat. No. 10,029,993, the disclosure of which is incorporated by reference herein. In embodiments, the CYP11β2 beta hydroxylase inhibitor is a compound described in U.S. Pat. No. 10,329,263, the disclosure of which is incorporated by reference herein. In embodiments, the CYP 11β2 beta hydroxylase inhibitor is a 1,2,4-triazine compound or a pharmaceutically acceptable salt thereof. In embodiments, the CYP11β2 beta hydroxylase inhibitor is a compound of Formula (A) or a pharmaceutically acceptable salt thereof. In embodiments, the CYP11β2 beta hydroxylase inhibitor is a pharmaceutically acceptable salt of the compound of Formula (A). In embodiments, the CYP11β2 beta hydroxylase inhibitor is a monohydrobromide salt of the compound of Formula (A). The provided pharmaceutical compositions are suitable for administration in the methods described herein. Suitable excipients are described in Remington: The Science and Practice of Pharmacy, 21st Edition, David B. Troy, ed., Lippicott Williams & Wilkins (2005).

“Pharmaceutically acceptable excipient” refer to a substance that aids the administration of an active agent to and absorption by a subject and can be included in the compositions of the disclosure without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the disclosure. One of skill in the art will recognize that other pharmaceutical excipients are useful.

Solutions of the active compounds as free base or pharmacologically acceptable salt can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations can contain a preservative to prevent the growth of microorganisms.

Pharmaceutical compositions can be delivered via intranasal or inhalable solutions or sprays, aerosols or inhalants. Nasal solutions can be aqueous solutions designed to be administered to the nasal passages in drops or sprays. Nasal solutions can be prepared so that they are similar in many respects to nasal secretions. Thus, the aqueous nasal solutions usually are isotonic and slightly buffered to maintain a pH of 5.5 to 6.5. In addition, antimicrobial preservatives, similar to those used in ophthalmic preparations and appropriate drug stabilizers, if required, may be included in the formulation. Various commercial nasal preparations are known and can include, for example, antibiotics and antihistamines.

Oral formulations can include excipients as, for example, pharmaceutical grades of mannitol, lactose, starch, magnesium stearate, sodium saccharine, cellulose, magnesium carbonate and the like. These compositions take the form of solutions, suspensions, tablets, pills, capsules, sustained release formulations or powders. In embodiments, oral pharmaceutical compositions will comprise an inert diluent or edible carrier, or they may be enclosed in hard or soft shell gelatin capsule, or they may be compressed into tablets, or they may be incorporated directly with the food. For oral therapeutic administration, the active compounds may be incorporated with excipients and used in the form of ingestible tablets, buccal tablets, troches, capsules, elixirs, suspensions, syrups, wafers, and the like. The percentage of the compositions and preparations may, of course, be varied and may conveniently be between about 1 to about 80% of the weight of the unit. The amount of active compounds in such compositions is such that a suitable dosage can be obtained.

For parenteral administration in an aqueous solution, for example, the solution should be suitably buffered and the liquid diluent first rendered isotonic with sufficient saline or glucose. Aqueous solutions, in particular, sterile aqueous media, are especially suitable for intravenous, intramuscular, subcutaneous and intraperitoneal administration. For example, one dosage could be dissolved in 1 ml of isotonic NaCl solution and either added to 1000 ml of hypodermoclysis fluid or injected at the proposed site of infusion.

Sterile injectable solutions can be prepared by incorporating the active compounds in the required amount in the appropriate solvent followed by filtered sterilization. Generally, dispersions are prepared by incorporating the various sterilized active ingredients into a sterile vehicle which contains the basic dispersion medium. Vacuum-drying and freeze-drying techniques, which yield a powder of the active ingredient plus any additional desired ingredients, can be used to prepare sterile powders for reconstitution of sterile injectable solutions. The preparation of more, or highly, concentrated solutions for direct injection is also contemplated. Dimethyl sulfoxide can be used as solvent for rapid penetration, delivering high concentrations of the active agents to a small area.

The formulations of compounds can be presented in unit-dose or multi-dose sealed containers, such as ampules and vials. Thus, the composition can be in unit dosage form. In such form the preparation is subdivided into unit doses containing appropriate quantities of the active component. Thus, the compositions can be administered in a variety of unit dosage forms depending upon the method of administration. For example, unit dosage forms suitable for oral administration include, but are not limited to, powder, tablets, pills, capsules and lozenges.

In embodiments, the compositions described herein comprise a dose of the CYP11β2 beta hydroxylase inhibitor in an amount from about 0.001 mg to about 1,000 mg. In embodiments, the dose is from about 0.01 mg to about 900 mg. In embodiments, the dose is from about 0.1 mg to about 800 mg. In embodiments, the dose is from about 1 mg to about 700 mg. In embodiments, the dose is from about 1 mg to about 600 mg. In embodiments, the dose is from about 1 mg to about 500 mg. In embodiments, the dose is from about 1 mg to about 400 mg.

The frequency (e.g., once per day, twice per day, thrice per day) and duration (e.g., one week, two weeks, one month, two months, six months, 1 year, 5 to 10 years, or until disease progression) of administration of the CYP11β2 beta hydroxylase inhibitor or the pharmaceutical compositions described herein can vary depending upon a variety of factors, for example, whether the patient suffers from another disease, and the route of administration; size, age, sex, health, body weight; nature and extent of symptoms of the disease being treated; whether there is concurrent treatment, complications from the disease being treated or other health-related problems. Adjustment and manipulation of the frequency and duration of treatment are within the ability of one skilled in the art. In embodiments, the CYP11β2 beta hydroxylase inhibitor or the pharmaceutical compositions described herein are administered to the patient once per day. In embodiments, the CYP11β2 beta hydroxylase inhibitor or the pharmaceutical compositions are administered twice per day. In embodiments, the CYP11β2 beta hydroxylase inhibitor or pharmaceutical compositions are administered thrice per day.

In embodiments, the methods for treating hypertension further comprise administering a second agent (e.g. therapeutic agent). In embodiments, the methods include administering a second agent (e.g. therapeutic agent) in a therapeutically effective amount. In embodiments, the second agent is a hypertension medication. In embodiments, the second agent is a diuretic, an ACE inhibitor, an angiotensin receptor blocker, a calcium channel blocker, or a combination of two or more thereof.

### General

All combinations of the various elements disclosed herein are within the scope of the invention. For example, within the scope of the invention are embodiment in which any threshold level described herein may be combined with any other threshold level described herein.

As used herein, all headings are simply for organization and are not intended to limit the disclosure in any manner. The content of any individual section may be equally applicable to all sections. All combinations of the various elements disclosed herein are within the scope of the invention.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

### EXAMPLES

The following examples are for purposes of illustration and are not intended to limit the spirit or scope of the disclosure or claims.

### Example 1

The identification of patients with low renin hypertension for a novel targeted therapy using an aldosterone synthase inhibitor will maximize the benefit:risk ratio of a new CYP11β2 beta hydroxylase inhibitor. In practice, the patients will be identified and selected from among individuals with diagnosed essential hypertension (also known as primary hypertension) in stepwise fashion. First by measuring the patient's systolic and diastolic blood pressure. The first selection criteria will be systolic blood pressure of greater than 140 mmHg and/or diastolic blood pressure of greater than 90 mmHg, e.g., as defined by 2018 ESH-ESC Guidelines (Williams et al, 2018 Practice Guidelines for the management of arterial hypertension of the European Society of Hypertension and the European Society of Cardiology: ESH/ESC Task Force for the Management of Arterial Hypertension [published correction appears in J Hypertens. 2019 February; 37(2):456]. J Hypertens. 2018; 36(12):2284-2309), measured by Automated Office Blood Pressure Measurement. (SPRINT Research Group, Wright J T Jr, Williamson J D, et al. A Randomized Trial of Intensive versus Standard Blood-Pressure Control [published correction appears in N Engl J Med. 2017 Dec. 21; 377(25):2506]. N Engl J Med. 2015; 373(22):2103-2116). Among these individuals, patients who are taking two or more blood pressure medications from standard-of-care hypertension medications (e.g., diuretic, ACE inhibitor, angiotensin receptor blocker, calcium channel blocker) at their maximum doses and/or maximum tolerated doses will be selected. Patients in this group will then be assessed for plasma concentrations of two hormones in the hypothalamic-pituitary-adrenal axis, renin and aldosterone. Using a commercially available, FDA-approved laboratory assay, a value of plasma renin activity less than or equal to 0.6 units/milliliter/hour taken as a 24-hour sample will be considered one selection criterion. Using a commercially available, FDA-approved laboratory assays, a value of plasma aldosterone concentration of greater than or equal to 6 ng/dL will be considered the second hormonal criterion.

The subset of essential hypertension patients emerging from this selection process will be predisposed favorably to treatment with selective inhibitors of aldosterone synthase (e.g., CYP 11β2 beta hydroxylase). After receiving treatment with a selective inhibitor of aldosterone synthase (e.g., the compound of Formula (A) or a pharmaceutically acceptable salt thereof (e.g., monohydrobromide salt)) these patients will exhibit, e.g., acute and long-term improvement in blood pressure (e.g., systolic and/or diastolic). These patients will also exhibit a diminished risk and manifestation of morbidities, including damage and dysfunction of the cardiac and renovascular organ systems, among others.

### Example 2

Aldosterone is produced by an aldosterone synthase, encoded by the gene CYP11B2. Aldosterone synthase catalyzes the final 3 steps in aldosterone synthesis, sequentially (1) from 11-deoxycorticosterone (11-DOC) to corticosterone, (2) from corticosterone to 18-OH-corticosterone, and (3) from 18-OH-corticosterone to aldosterone. Notably, aldosterone synthase shares a high homology to 11β-hydroxylase, encoded by the gene CYP11B1, which is responsible for cortisol production. 11β-hydroxylase catalyzes the conversion of 11-deoxycortisol to cortisol, and 11-deoxycorticosterone (11-DOC) to corticosterone.

Because 11-DOC is a substrate for both CYP11B1 and CYP11B2, use of a selective aldosterone synthase inhibitor which preserves the activity of CYP11B1 is important for preventing accumulation of the precursor 11-DOC. As shown in this example, the compound of Formula (A) is selective, as it exhibits a wide does range at which aldosterone is suppressed without an increase in 11-DOC or change in cortisol production.

Single Ascending Dose (SAD) and Multiple Ascending Dose (MAD) Study

A randomized, double-blinded, placebo-controlled study was conducted in which 116 patients were randomized and 87 received the compound of Formula A in the form of an HBr salt. As discussed below and shown in the accompanying figures, the study demonstrated rapid and durable reduction/suppression of plasma aldosterone with no effect on cortisol levels. No deaths or serious adverse events (SAEs) occurred. Further, adverse events (AEs) were comparable between the groups receiving the compound and placebo groups. No clinically significant findings with respect to clinical laboratory, vital signs, ECGs or physical examination were reported. No hyperkalemia was noted.

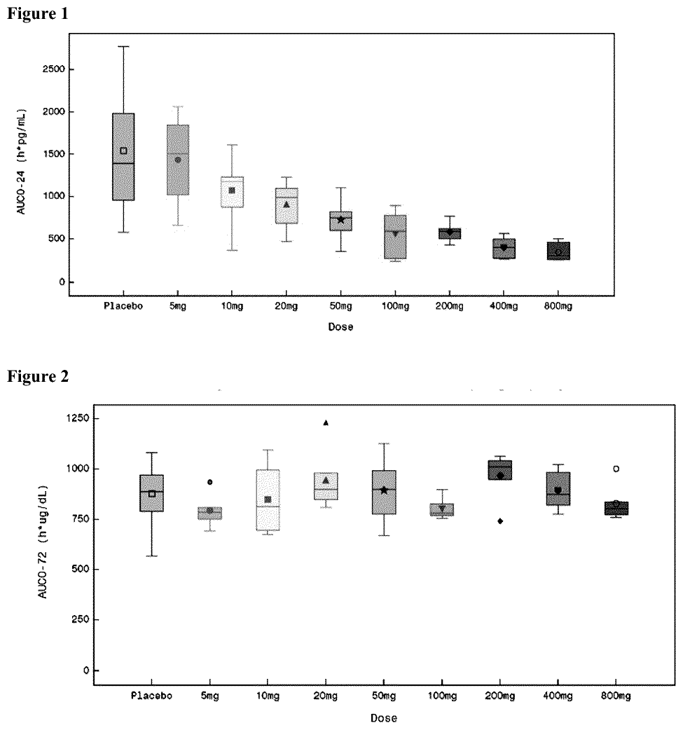

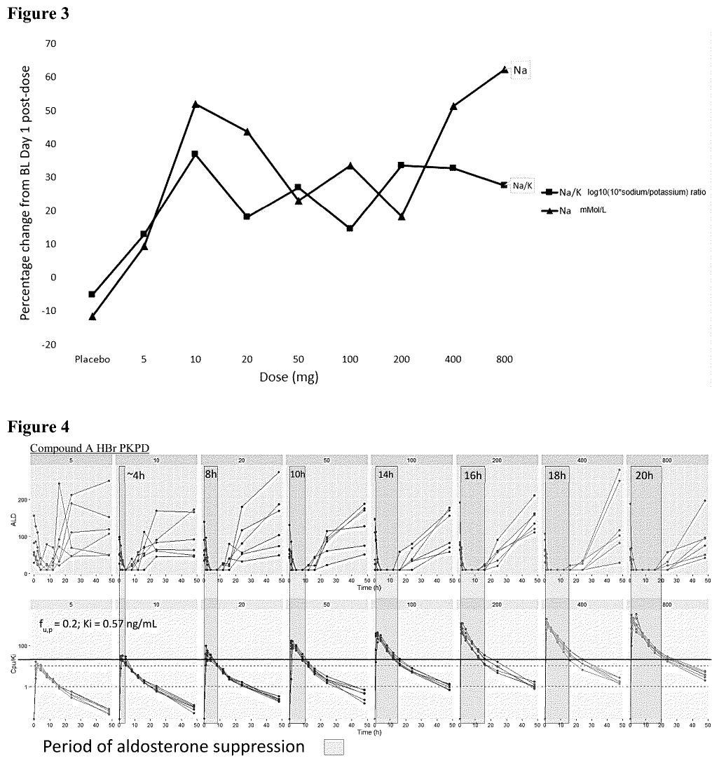

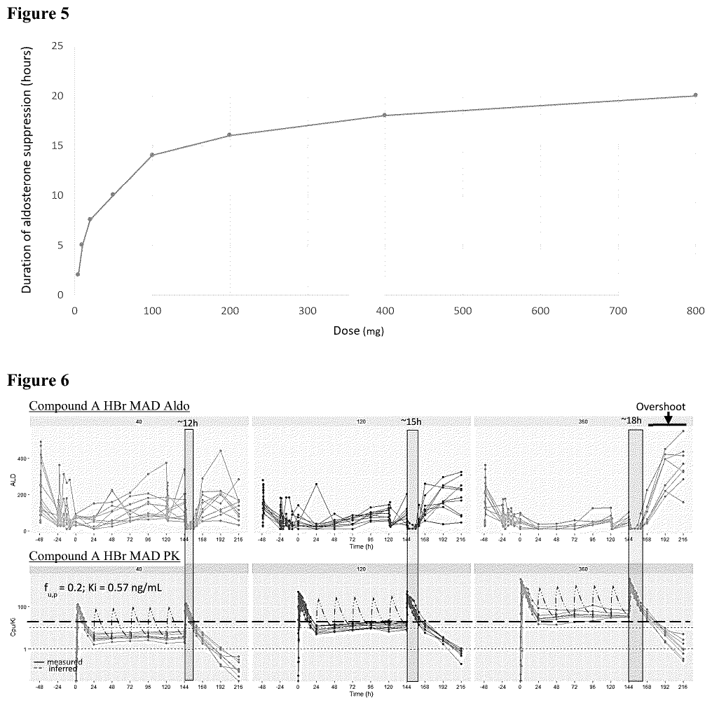

In the SAD study, a 50% reduction of serum aldosterone at a dose of 50 mg was observed (FIG. 1) and there was no evidence of dose-dependent reduction in serum cortisol (FIG. 2). Further, at doses greater than 10 mg, similar magnitudes of naturesis in post-dose spot urine were observed (FIG. 3). Exposure above about 3-4 times the inhibition constant (Ki) was associated with duration of aldosterone suppression (FIG. 4). A dose-dependent increase in duration of maximum aldosterone suppression was observed (FIG. 5).

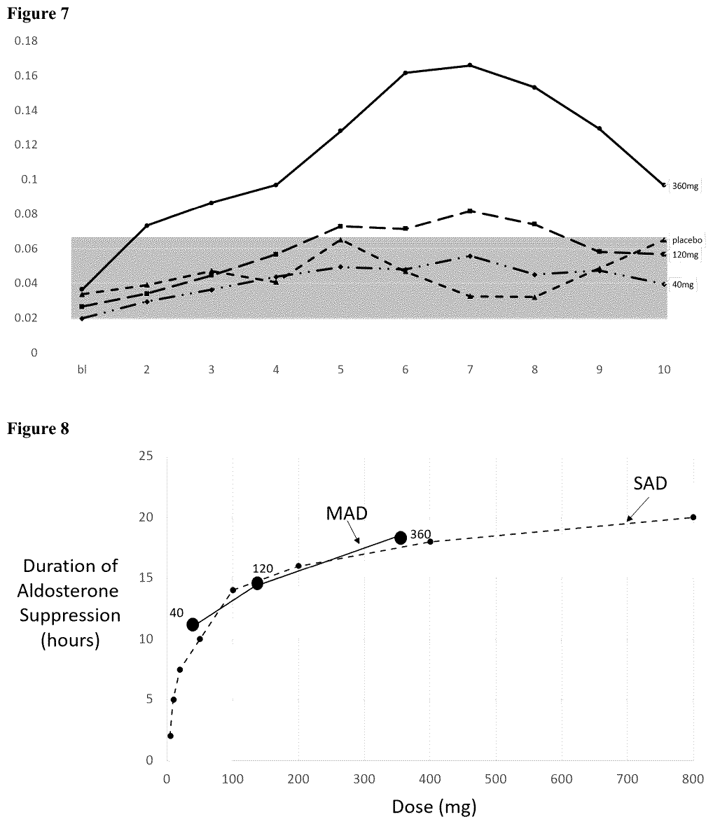

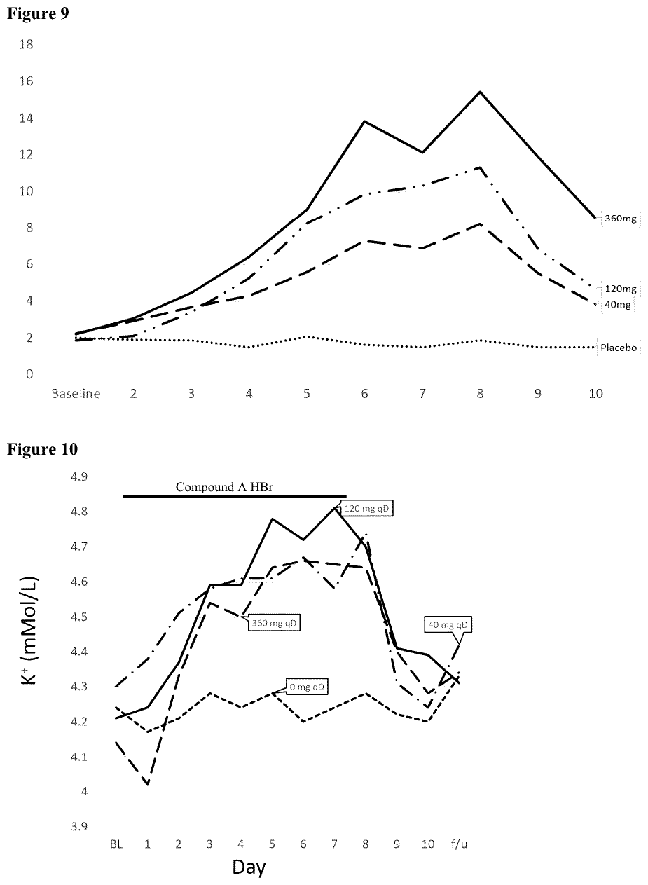

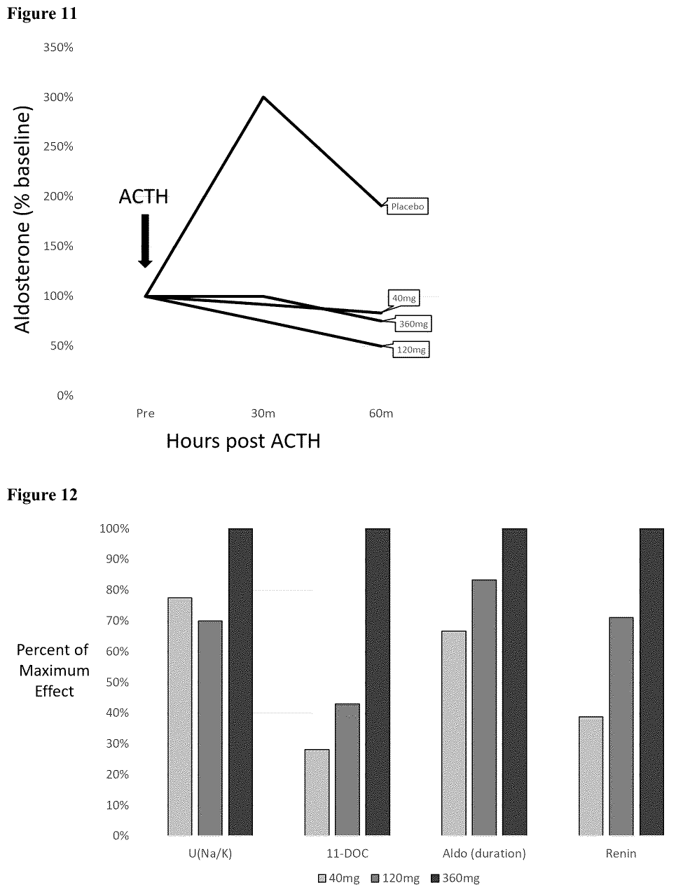

In the MAD study, little drug accumulation was observed. At the 360 mg dose, an overshoot in serum aldosterone was observed during the washout phase, consistent with accumulation of 11-DOC during treatment (FIG. 6). Accumulation of 11-DOC at the 360 mg dose level likely underlies the observed overshoot in aldosterone production after discontinuation of administration of the compound on day 7 (FIG. 7). The dose-effect of the compound on duration of maximum aldosterone suppression was consistent with the SAD study, with little drug accumulation and comparable duration of aldosterone to SAD on day 7 (FIG. 8). A dose-dependent increase in Plasma Renin Activity (PRA) was observed with 50% of maximum at the 40 mg dose (FIG. 9). With multiple dosing, a physiological, non-adverse, increase in K⁺ (FIG. 10) and suppression of adrenocorticotropic hormone (ACTH)-stimulated aldosterone secretion (FIG. 11) was observed at all doses tested. Benefits on renal tubular sodium excretion were seen at lower doses than are required for stimulation of renin and accumulation of 11-DOC (FIG. 12).

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### Example 3

Purpose: To determine whether CYP11B2 blockade (via aldosterone synthase inhibition) improves cardiovascular function in an animal model for low renin hypertension.

Animal Model: All experiments were conducted in 12-16-week-old obese AY female mice. All animals exhibited hypertension and exhibited vascular dysfunction at this age.

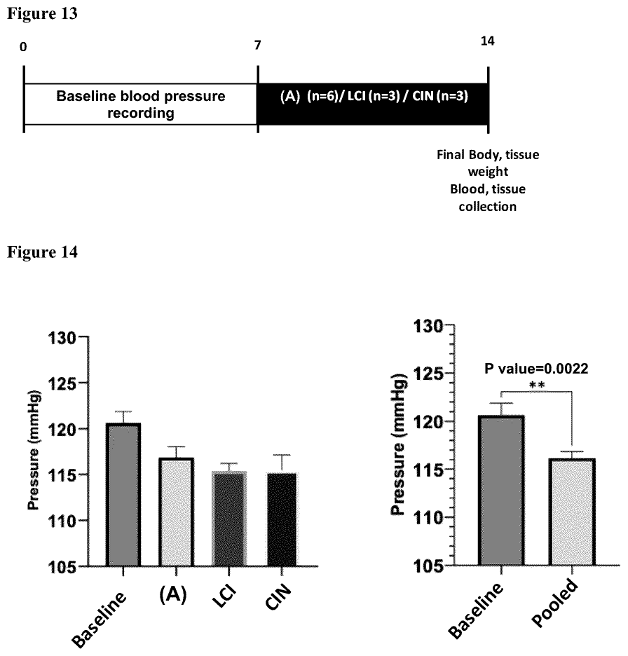

Telemetry Protocol: The telemetry protocol is illustrated in FIG. 13.

Treatment Protocol: Male and female Balb/C mice (Jackson Labs, Bar Harbor, ME, Catalog #000651) at 10 weeks of age were utilized for all studies. Mice were implanted with indwelling telemeters (carotid catheter) (DSI© Model #PA-C10, New Brighton, MN) under isoflurane anesthesia as previously described. Following a 7-day recovery period baseline BP measurements were consciously recorded for 7 days while mice were on normal salt diet (NS) (0.2% NaCl, Teklad #2918, Envigo, United Kingdom). Mice were then treated with the test article (compound of formula (A), LCI699 or CIN-107, calculated to deliver 3 mg/kg/day) infused via an implanted mini-osmotic pump for an additional 7 days.

Blood pressure measurement: For each protocol described in FIG. 13, 12 mice were used to record systolic, diastolic, mean arterial pressure heart rate and activity via radio-telemetry. Telemetry transmitters were implanted in the carotid artery at 12 weeks of age. After 2 weeks of recovery from surgery, baseline blood pressure was recorded for 7 consecutive days. Animals were then submitted to the treatment (compound of formula (A) HBr/LCI/CIN).

Quantification of the effects of compound of formula (A) HBr, LCI, CIN on the degree of RAAS activation: Blood was collected from all the treated mice to investigate the effects of the aldosterone synthase inhibitors on plasma aldosterone, Angiotensin II and indices of RAS activation via LC-MS/MS.

Results: FIG. 14 shows the mean arterial blood pressure (MAP) in treated and untreated agouti yellow obese hyperleptinemic mice (Ay). Ay mice have approximately 5-8 mmHg elevation in MAP when compared to wild-type mice, due to direct leptin-mediated elevation in Cyp11β2 activity and aldosterone overproduction independent of RAS pathway activation (Hypertension, 2016 May; 67(5): 1020-1028; Circulation 2015 December 132(22); 2134-2145). Mice were treated with one of three Cyp11β2 inhibitors: the compound of formula (A) HBr as described herein; LCI699 or CIN-107. All three inhibitors reduced MAP to a similar degree and to a value comparable to that seen in control mice. The pooled data, comparing baseline to treated MAP in inhibitor-treated mice demonstrated statistically significant reduction (p=0.0022) verifying the ability of Cyp11β2 inhibitors to reduce blood pressure in the setting of aldosterone-mediated, Renin-angiotension-independent hypertension. There were no statistically significant differences in the treatment effect between the three inhibitors.

What is claimed is:

1. A method of treating hypertension in a low renin hypertensive subject, the method comprising administering to the low renin hypertensive subject an effective amount of a CYP 11β2 beta hydroxylase inhibitor.

2. The method of claim 1, wherein said low renin hypertensive subject is taking or has taken a hypertension medication selected from a diuretic, an ACE inhibitor, an angiotensin receptor blocker, a calcium channel blocker, or a combination of two or more thereof.

3. The method of claim 2, wherein the low renin hypertensive subject is taking or has taken at least two of said hypertension medications.

4. The method of claim 1, wherein the low renin hypertensive subject has a plasma renin activity less than or equal to 0.6 units/milliliter/hour.

5. The method of any one of claims 2 to 3, wherein the low renin hypertensive subject has a plasma renin activity less than or equal to 1 unit/milliliter/hour.

6. The method of any one of claims 1 to 5, wherein the low renin hypertensive subject has a plasma aldosterone concentration of greater than or equal to 6 ng/dL as measured by an immunoassay.

7. The method of any one of claims 1 to 5, wherein the low renin hypertensive subject has a plasma aldosterone concentration of greater than or equal to 1 ng/dL as measured by LC-MS.

8. The method of any one of claims 1 to 7, wherein said CYP 11β2 beta hydroxylase inhibitor is selective for inhibition of CYP 11β2 beta hydroxylase activity relative to inhibition of CYP 11β1 beta hydroxylase activity, preferably wherein the inhibition constant (Ki) for CYP 11β1 beta hydroxylase divided by the Ki for CYP 11β2 beta hydroxylase is greater than 100.

9. The method of any one of claims 1 to 8, wherein said CYP 11β2 beta hydroxylase inhibitor is a 1,2,4-triazine compound or a pharmaceutically acceptable salt thereof.

10. The method of any one of claims 1 to 9, wherein said CYP 11β2 beta hydroxylase inhibitor is a compound of formula (I) or a pharmaceutically acceptable salt thereof: 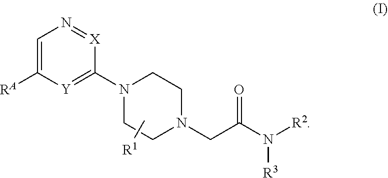
(i) wherein X and Y represent any of the following (i) to (iii):
(a) X is N, and Y is CH or C—RY,
(b) X is CH, and Y is N, or
(c) X is CH, and Y is CH;
(ii) R^{Y} represents an alkyl group;
(iii) R^{A} represents a cycloalkyl group which may be substituted, a cycloalkenyl group which may be substituted, an aryl group which may be substituted, or a 6- to 10-membered monocyclic or bicyclic heteroaryl group which may be partially hydrogenated and may be substituted;
(iv) R¹ represents a hydrogen atom, or an alkyl group;
(v) R² represents an alkyl group which may be substituted, a cycloalkyl group which may be substituted, an aliphatic heterocyclic group which may be substituted, or a heteroaryl group which may be partially hydrogenated and may be substituted; and
(vi) R³ represents a hydrogen atom, or an alkyl group, or a pharmaceutically acceptable salt thereof.

11. The method of any one of claims 1 to 9, wherein said CYP 11β2 beta hydroxylase inhibitor is a compound of Formula (A) or a pharmaceutically acceptable salt thereof:

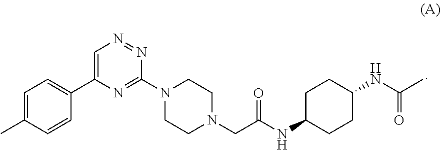 [SMILES: CC(=O)N[C@H]1CC[C@H](NC(=O)CN2CCN(c3nncc(-c4ccc(C)cc4)n3)CC2)CC1]

12. The method of claim 11, wherein the compound is in the form of an HBr salt of the compound of Formula (A).

13. The method of claim 11 or 12, wherein:
(i) between 5 mg and 100 mg of the CYP 11β2 beta hydroxylase inhibitor is administered orally twice a day, 12 hours apart;
(ii) between 10 mg and 50 mg of the CYP 11β2 beta hydroxylase inhibitor is administered orally twice a day, 12 hours apart;
(iii) between 5 mg and 100 mg of the CYP 11β2 beta hydroxylase inhibitor is administered orally once a day; or
(iv) between 10 mg and 50 mg of the CYP 11β2 beta hydroxylase inhibitor is administered orally once a day.

14. The method of claim 11 or 12, wherein:
(i) 12.5 mg of the CYP 11β2 beta hydroxylase inhibitor is administered orally twice a day, 12 hours apart;
(ii) 25 mg of the CYP 11β2 beta hydroxylase inhibitor is administered orally twice a day, 12 hours apart;
(iii) 12.5 mg of the CYP 11β2 beta hydroxylase inhibitor is administered orally once a day;
(iv) 50 mg of the CYP 11β2 beta hydroxylase inhibitor is administered orally once a day; or
(v) 100 mg of the CYP 11β2 beta hydroxylase inhibitor is administered orally once a day.

15. The method of any one of claims 1 to 14, wherein the CYP 11β2 beta hydroxylase inhibitor does not inhibit the activity of 11β-hydroxylase in the subject as demonstrated by a lack of a clinically meaningful reduction in cortisol production in an ACTH (Cortrosyn) Stimulation test, preferably wherein the post-stimulation cortisol levels are greater 18 mcg/dL.

16. The method of any one of claims 1 to 15, wherein the CYP 11β2 beta hydroxylase inhibitor is administered to the low renin hypertensive subject in an amount which:
(i) suppresses aldosterone production in the subject;
(ii) increases serum and/or plasma potassium levels in the subject; and/or
(iii) increases plasma renin activity (PRA) in the subject.

17. The method of any one of claims 1 to 16, wherein the CYP 11β2 beta hydroxylase inhibitor is administered to the low renin hypertensive subject in an amount below the amount which causes the subject's serum and/or plasma 11-deoxycortisterone (11-DOC) levels to exceed 600 pmol/L, preferably below the amount which causes the subject's serum and/or plasma 11-DOC levels to exceed 400 pmol/L.

18. The method of any one of claims 1 to 17, wherein the CYP 11β2 beta hydroxylase inhibitor is administered to the low renin hypertensive subject in an amount below the amount which causes an accumulation of 11-DOC above 0.1 ng/ml in the subject.

19. The method of any of claims 16 to 18, wherein:
(i) serum and/or plasma aldosterone AUC-24 is reduced in the subject by at least 25% relative to the aldosterone levels in the subject prior to administration of the CYP 11β2 beta hydroxylase inhibitor;
(ii) serum and/or plasma potassium levels in the subject are increased by at least 0.3 mMol/L relative to the serum and/or plasma potassium levels in the subject prior to administration of the CYP 11β2 beta hydroxylase inhibitor; and/or
(iii) PRA in the subject is increased by at least 5 ng/nl/hr relative to the PRA in the subject prior to administration of the CYP 11β2 beta hydroxylase inhibitor.

20. The method of any one of claims 1 to 19, wherein the CYP 11β2 beta hydroxylase inhibitor is administered to the low renin hypertensive subject in an amount which does not cause a clinically meaningful upregulation of the subject's adrenocortical hormone synthesis.

21. The method of any one of claims 1 to 20, wherein the administration of the CYP 11β2 beta hydroxylase inhibitor is administered to the low renin hypertensive subject in an amount which:
(i) does not cause a clinically meaningful reduction of the subject's serum and/or plasma cortisol levels, relative to the subject's serum and/or plasma cortisol levels prior to administration of the CYP 11β2 beta hydroxylase inhibitor;
(ii) does not cause a clinically meaningful increase in the subject's serum and/or plasma 11-DOC levels relative to the subject's serum and/or plasma 11-DOC levels prior to administration of the CYP 11β2 beta hydroxylase inhibitor; and/or
(iii) does not cause a clinically meaningful increase in the subject's serum and/or plasma 11-deoxycortisol levels relative to the subject's serum and/or plasma 11-deoxycortisol levels prior to administration of the CYP 11β2 beta hydroxylase inhibitor.

22. The method of any one of claims 1 to 21, wherein the CYP 11β2 beta hydroxylase inhibitor is administered to the low renin hypertensive subject in an amount:
(i) which does not cause a reduction of more than 20% in the subject's serum and/or plasma cortisol levels, relative to the subject's serum and/or plasma cortisol levels prior to administration of the CYP 11β2 beta hydroxylase inhibitor, preferably which does not cause a reduction of more than 10% in the subject's serum and/or plasma cortisol levels, relative to the subject's serum and/or plasma cortisol levels prior to administration of the CYP 11β2 beta hydroxylase inhibitor;
(ii) which does not cause an increase of more than 20% in the subject's serum and/or plasma 11-DOC levels relative to the subject's serum and/or plasma 11-DOC levels prior to administration of the CYP 11β2 beta hydroxylase inhibitor, preferably which does not cause an increase of more than 10% in the subject's serum and/or plasma 11-DOC levels relative to the subject's serum and/or plasma 11-DOC levels prior to administration of the CYP 11β2 beta hydroxylase inhibitor; and/or
(iii) which does not cause an increase of more than 20% in the subject's serum and/or plasma 11-deoxycortisol levels relative to the subject's serum and/or plasma 11-deoxycortisol levels prior to administration of the CYP 11β2 beta hydroxylase inhibitor, preferably which does not cause an increase of more than 10% in the subject's serum and/or plasma 11-deoxycortisol levels relative to the subject's serum and/or plasma 11-deoxycortisol levels prior to administration of the CYP 11β2 beta hydroxylase inhibitor.

23. The method of any one of claims 1 to 22, wherein:
(i) the subject's office-measured systolic blood pressure is lowered relative to the subject's office-measured systolic blood pressure prior to administration of the CYP 11β2 beta hydroxylase inhibitor; and/or
(ii) the subject's ambulatory systolic blood pressure as measured by automated office blood pressure measurement (AOB) or sphygmomanometer is lowered relative to the subject's ambulatory systolic blood pressure prior to administration of the CYP 11β2 beta hydroxylase inhibitor.

24. The method of claim 23, wherein:
(i) the subject's office-measured systolic blood pressure is lowered by at least 10 mmHg relative to the subject's office-measured systolic blood pressure prior to administration of the CYP 11β2 beta hydroxylase inhibitor; and/or
(ii) the subject's ambulatory systolic blood pressure is lowered by at least 10 mmHg relative to the subject's ambulatory systolic blood pressure prior to administration of the CYP 11β2 beta hydroxylase inhibitor.

25. The method of any one of claims 1 to 24, wherein:
(i) the subject's ambulatory systolic and diastolic blood pressure is lowered relative to the subject's ambulatory systolic and diastolic blood pressure prior to administration of the CYP 11β2 beta hydroxylase inhibitor;
(ii) the subject's office-measured systolic and diastolic blood pressure is lowered relative to the subject's office-measured systolic and diastolic blood pressure prior to administration of the CYP 11β2 beta hydroxylase inhibitor;
(iii) the subject's office-measured diastolic blood pressure is lowered relative to the subject's office-measured diastolic blood pressure prior to administration of the CYP 11β2 beta hydroxylase inhibitor; and/or
(iv) the subject's systolic blood pressure is reduced to less than 130 mmHg and/or the subject's diastolic blood pressure is reduced to less than 80 mmHg.

26. The method of claim 25, wherein:
(i) the subject's ambulatory systolic blood pressure is lowered by at least 10 mmHg and the subject's ambulatory diastolic blood pressure is lowered by at least 5 mmHg, each relative to the subject's ambulatory systolic and diastolic blood pressure, respectively, prior to administration of the CYP 11β2 beta hydroxylase inhibitor;
(ii) the subject's office-measured systolic blood pressure is lowered by at least 10 mmHg and the subject's office-measured diastolic blood pressure is lowered by at least 5 mmHg, each relative to the subject's office-measured systolic and diastolic blood pressure, respectively, prior to administration of the CYP 11β2 beta hydroxylase inhibitor;
(iii) the subject's office-measured diastolic blood pressure is lowered by at least 5 mmHg relative to the subject's office-measured diastolic blood pressure prior to administration of the CYP 11β2 beta hydroxylase inhibitor; and/or
(iv) the subject's systolic blood pressure is reduced to less than 130 mmHg and/or the subject's diastolic blood pressure is reduced to less than 80 mmHg.

27. The CYP 11β2 beta hydroxylase inhibitor or composition recited in any one of claims 1 to 14 for use in treating a low renin hypertensive subject.

28. The CYP 11β2 beta hydroxylase inhibitor or composition of claim 27, wherein:
(i) said low renin hypertensive subject is taking or has taken a hypertension medication selected from a diuretic, an ACE inhibitor, an angiotensin receptor blocker, a calcium channel blocker, or a combination of two or more thereof;
(ii) wherein the low renin hypertensive subject is taking or has taken at least two of said hypertension medications;
(iii) the low renin hypertensive subject has a plasma renin activity less than or equal to 0.6 units/milliliter/hour when not taking a hypertension medication or a plasma renin activity less than or equal to 1 unit/milliliter/hour when taking one or more hypertension medications; and/or
(iv) the low renin hypertensive subject has a plasma aldosterone concentration of greater than or equal to 6 ng/dL as measured by an immunoassay or greater than or equal to 1 ng/dL as measured by LC-MS.

29. The CYP 11β2 beta hydroxylase inhibitor or composition of claim 27 or 28, wherein, when administered to a low renin hypertensive subject, the low renin hypertensive subject experiences one or more or all of the effects recited in claims 15 to 26.

30. A method of identifying a subject for hypertension treatment with a CYP 11β2 beta hydroxylase inhibitor, the method comprising:
(i) measuring a systolic blood pressure of greater than 130 mmHg in said subject;
(ii) measuring a diastolic BP of greater than 90 mmHg in said subject;
(iii) determining that the subject has a plasma renin activity of less than or equal to 0.6 units/milliliter/hour when not taking a hypertension medication or a plasma renin activity less than or equal to 1 unit/milliliter/hour when taking one or more hypertension medications; and
(iv) determining that the subject has a plasma aldosterone concentration of greater than or equal to 6 ng/dL if measured by an immunoassay;
thereby identifying said subject for hypertension treatment with a CYP 11β2 beta hydroxylase inhibitor.

31. The method of claim 30, wherein step (iv) comprises determining that the subject has a plasma aldosterone concentration of greater than or equal to 1 ng/dL if measured by LC-MS.

32. A method of treating hypertension in a subject in need thereof, the method comprising:
(i) measuring a systolic blood pressure of greater than 130 mmHg in said subject;
(ii) measuring a diastolic BP of greater than 90 mmHg in said subject;
(iii) determining that the subject has a plasma renin activity less than or equal to 0.6 units/milliliter/hour when not taking a hypertension medication or a plasma renin activity less than or equal to 1 unit/milliliter/hour when taking one or more hypertension medications;
(iv) determining that the subject has a plasma aldosterone concentration of greater than or equal to 6 ng/dL if measured by an immunoassay; and
(v) administering to said subject an effective amount of a CYP 11β2 beta hydroxylase inhibitor.

33. The method of any one of claims 30 to 32, wherein step (iv) comprises determining that the subject has a plasma aldosterone concentration of greater than or equal to 1 ng/dL if measured by LC-MS.

34. The method of any one of claims 30 to 33, wherein said CYP 11β2 beta hydroxylase is selective for inhibition of CYP 11β2 beta hydroxylase activity relative to inhibition of CYP 11β1 beta hydroxylase activity, preferably wherein the inhibition constant (Ki) for CYP 11β1 beta hydroxylase divided by the Ki for CYP 11β2 beta hydroxylase is greater than 100.

35. The method of any one of claims 30 to 34, wherein said CYP 11β2 beta hydroxylase inhibitor is a 1,2,4-triazine compound or a pharmaceutically acceptable salt thereof.

36. The method any one of claims 30 to 35, wherein said CYP 11β2 beta hydroxylase inhibitor is a compound of Formula (A) or a pharmaceutically acceptable salt thereof: 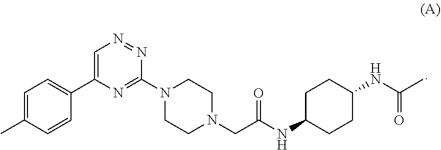 [SMILES: CC(=O)N[C@H]1CC[C@H](NC(=O)CN2CCN(c3nncc(-c4ccc(C)cc4)n3)CC2)CC1]
